(19) United States
(12) Patent Application Publication
(10) Pub. No.: US 2025/0166746 A1
(43) Pub. Date: May 22, 2025

(54) SYSTEMS AND METHODS FOR DESIGNING RANDOMIZED CONTROLLED STUDIES

(71) Applicant: OWKIN, INC., New York, NY (US)

(72) Inventors: Félix Balazard, Paris (FR); Raphaëlle Momal, Paris (FR); Paul Trichelair, Paris (FR); Michael Blum, Paris (FR)

(21) Appl. No.: 18/843,606

(22) PCT Filed: Mar. 6, 2023
(86) PCT No.: PCT/US2023/014623

§ 371 Date: Sep. 3, 2024

(30) Foreign Application Priority Data
Mar. 4, 2022 (EP) 22305252.3

(51) Int. Cl. G16H 10/20; G06T 7/00; G06V 10/44; G06V 10/82; G06V 20/69; G16H 50/30

(52) U.S. Cl. CPC G16H 10/20; G06T 7/0012; G06V 10/44; G06V 10/82; G06V 20/695; G06V 20/698; G16H 50/30; G06T 2207/10056; G06T 2207/20021; G06T 2207/20084; G06T 2207/30024

(57) ABSTRACT

Methods for designing a study, e.g., randomized controlled trial (RCT), and methods for evaluating sample size in an uncommenced or ongoing study are provided. Also provided are methods for conducting co-variate adjustment using covariates (e.g., prognostic covariates) obtained by deep learning models in a study.

### FIELD OF INVENTION

This invention relates generally to adjustment of covariates and study design process.

### BACKGROUND OF THE INVENTION

In studies in which the goal is to test the presence of a treatment effect (bi), the relationship among outcome (Y), treatment allocation (T), and prognostic covariate (X) that is associated with Y can be provided for instance as:

$$Y = {b}_{0} + {b}_{1} T + {b}_{2} X + eps$$

where b₀, b₁, and b₂ denote coefficients and eps (epsilon) denotes error. In this formula, covariate adjustment (i.e., providing b₂X) allows for more precise estimation of a treatment effect (b₁).

Adjustment on prognostic covariates allows for improved precision and efficiency in analysis and increased statistical power for treatment effect estimation in studies, e.g., randomized clinical trials. Covariate adjustment enables achieving the same statistical power with a smaller sample size. Adjustment covariates should be prespecified and should be selected based on their prognostic value and not on any imbalance criterion. With recognition of the significance of covariate adjustment in a study, this methodological consensus has been implemented by regulatory agencies including the European Medical Association (EMA) and the Food and Drug Administration (FDA) as regulatory guidance. Achieving the same statistical power by using a smaller sample size and/or less strict eligibility criteria will enable reduction in size of the population that needs to be screened for enrollment in the study. Accordingly, there is a need for systems and methods for efficiently adjusting covariates, reducing the sample size, and/or relaxing the eligibility criteria in studies and improving efficiency of hypothesis testing.

### SUMMARY OF THE DESCRIPTION

The present disclosure provides methods for designing (e.g., reducing) sample size in a trial based on known correlation of the adjustment covariate and outcome, as well as methods for reestimating and readjusting sample size while maintaining the blindness of the trial, e.g., conducting blinded sample size reestimation, based on data that becomes available during the trial, both in the continuous and time-to-event outcome settings. The methods disclosed herein can incorporate novel sources of prognostic signals, e.g., prognostic scores obtained by deep learning, as adjustment covariates to compute the adjusted (e.g., reduced) sample size. The present disclosure also provides methods for achieving a targeted statistical power using less strict eligibility criteria in a trial by using covariate adjustment that can compensate for the increased heterogeneity that comes with less restrictive inclusion criteria.

In one aspect, the present disclosure provides a method for designing a randomized controlled trial (RCT) with a time-to-event outcome, said method comprising:
selecting a covariate for adjustment; and
calculating a number of events required to obtain a statistical power based on a formula:

N_{adjusted}=N_{original}(1−R_{CS}²); wherein
the RCT is conducted using the calculated number of events,
wherein the N_{original} is an original number of events required to obtain the statistical power without covariate adjustment,
wherein the N_{adjusted} is an adjusted number of events required to obtain the statistical power with covariate adjustment, and
wherein the R_{CS}² is computed on data external to the RCT based on a formula:

$${R}_{CS}^{2} = 1 - exp [ - \frac{2}{n} ( {l}_{1} - {l}_{0} ) ] ,$$

wherein
the R_{CS}² is a Cox-Snell R²,
the n is a number of participants,
the I₀ is a log-likelihood of a Cox model to explain the time-to-event outcome with an intercept only, and
the I₁ is a log-likelihood of a Cox model to explain the time-to-event outcome with an intercept and with covariate adjustment.

In another aspect, the present disclosure provides a method for evaluating sample size at an interim stage of an ongoing randomized controlled trial (RCT), said method comprising:
selecting a covariate for adjustment;
obtaining blinded RCT data; and
performing a blinded sample size reestimation, at the interim stage, using R_{CS}² and a formula:

N_{adjusted}=N_{original}(1−R_{CS}²); wherein
the RCT is further conducted using the blinded sample size reestimation,
wherein the N_{adjusted} is a reestimated number of events required to obtain the statistical power;
wherein the R_{CS}² is computed, at the interim stage, on the blinded RCT data based on a formula:

$${R}_{CS}^{2} = 1 - exp [ - \frac{2}{n} ( {l}_{1} - {l}_{0} ) ] ,$$

the R_{CS}² is a Cox-Snell R²,
the n is a number of participants,
the I₀ is a log-likelihood of a Cox model to explain the time-to-event outcome with an intercept only, and
the I₁ is a log-likelihood of a Cox model to explain the time-to-event outcome with an intercept and with covariate adjustment.

In some embodiments, the original number of events required to obtain the statistical power without covariate adjustment (N_{original}) is evaluated based on a formula:

N_{original}=(z_{β}+z_{1-α})²/(P₁P₂ log² (hr)), wherein
the N_{original} is an estimated number of events required to obtain the statistical power based on the Schoenfeld formula,
the α is a type I error level,
the β is a type II error level,
the P₁ and the P₂ are the proportion of the trial sample included in the treatment and control arm respectively (e.g. both are equal to ½ if the treatment allocation is balanced),
the hr is a stipulated hazard ratio, and
the z_{P} is a p-quantile of the standard normal distribution.

In some embodiments, the time-to-event outcome is overall survival, disease free survival, or time to disease relapse. In some embodiments, the RCT is conducted to evaluate a treatment effect in cancer patients. In some embodiments, the cancer is hepatocellular carcinoma, mesothelioma, pancreatic cancer, lung cancer, or breast cancer.

In some embodiments, covariate adjustment is conducted on a clinical risk score, said method comprising:
obtaining clinical attributes derived from the subject;
computing a clinical risk score using a clinical model, the clinical model trained using one or more subject attribute,
wherein the clinical risk score quantifies the prognosis of the subject.

In some embodiments, covariate adjustment is conducted on a covariate obtained by a deep learning model. In some embodiments, the deep learning model is based on histopathological slides obtained from cancer subjects, and the covariate is a prognostic covariate.

In some embodiments, the covariate is obtained by a computer-implemented method for determining a likelihood of prognosis of a subject having a disease, comprising:
accessing a digital histology image of a histology section obtained from the subject;
extracting a plurality of feature vectors of the histology image by applying a first convolutional neural network, wherein each of the features of the plurality of feature vectors represents local descriptors of the histology image;
classifying the histology image using at least the plurality of feature vectors and a classification model, wherein the classification model is trained using a training set of known histology images and known prognosis information; and
determining the likelihood of prognosis of the subject based on at least the classification of the histology image.

In some embodiments, the covariate is obtained by a computer-implemented method for determining the prognosis of a subject having a disease, said method comprising:
obtaining a digital histology image of a histology section from the subject;
dividing the digital image into a set of tiles;
extracting a plurality of feature vectors from the set of tiles, or a subset thereof; and
computing an artificial intelligence (AI) risk score based on the histology image using a machine learning model, the machine learning model having been trained by processing a plurality of training images to predict the prognosis,
wherein the Al risk score quantifies the prognosis of the subject.

In some embodiments, the method further comprises:
obtaining clinical attributes derived from the subject;
computing a clinical risk score using a clinical model, the clinical model trained using one or more subject attribute; and
computing a final risk score for the subject from the Al risk score and the clinical risk score,
wherein the final risk score quantifies the prognosis of the subject.

In some embodiments, the digital histology image is a whole slide image (WSI). In some embodiments, the histology section has been stained with a dye. In some embodiments, the dye is hematoxylin and eosin (H&E). In some embodiments, the disease is cancer, e.g., hepatocellular carcinoma, mesothelioma, pancreatic cancer, lung cancer, or breast cancer.

In some embodiments, subject enrollment based on restrictive eligibility criteria do not improve statistical power relative to subject enrollment based on less restrictive eligibility criteria. In some embodiments, a targeted statistical power is achieved using less strict eligibility criteria in a trial. In some embodiments, the method is implemented by a computer.

In some aspects, the present disclosure provides machine readable medium having executable instructions to cause one or more processing units to perform methods of designing a randomized controlled trial (RCT), or methods of evaluating sample size required to obtain a statistical power at an interim stage of an ongoing randomized controlled trial (RCT), as provided herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present invention is illustrated by way of example and not limitation in the figures of the accompanying drawings in which like references indicate similar elements.

FIG. 1 illustrates an exemplary workflow of designing a randomized trial and reestimating and readjusting the sample size to optimize efficiency and statistical power of the trial.

FIG. 2 illustrates an exemplary workflow of designing the sample size based on known correlation between the adjustment covariates and outcome.

FIG. 3 illustrates an exemplary workflow of blinded sample size reestimation in an ongoing trial.

FIG. 4 illustrates an exemplary workflow of parametric simulations. For a set of parameters corresponding to a clinical trial scenario, instances of clinical trials are simulated to allow for the comparison of the adjusted and unadjusted analysis.

FIGS. 5A-5C depict behavior of R_{obs}² on a range of C-index, the outcome incidence (λ), treatment effect (hr), Weibull shape (w), and drop-out rate (d). FIG. 5A: hr=0.7. FIG. 5B: hr=0.4. FIG. 5C: hr=0.7, d=0.01, w=0.5.

FIGS. 6A-6C depict relationships between proposed R2 measures and R_{obs}². FIG. 6A: w=0.5. FIG. 6B: w=1. FIG. 6C: w=1.5.

FIG. 7 depicts power curves resulting from adjustment on clinical variables only (tumor staging and ECOG score) and clinical variables and the additional deep learning covariate, HCCnet. Covariates are sampled from TCGA-HCC.

FIGS. 8A-8C depict power curves resulting from adjustment on histological subtype only (current trial) and histological subtype and the additional deep learning covariate, MesoNet (trial using MesoNet) for the PROMISE-meso trial (FIG. 8A), the BEAT-meso trial (FIG. 8B), the CheckMate 743 trial (FIG. 8C). All of these simulations were run using the MesoNet training dataset from the Mesobank.

FIGS. 9A and 9B depict interplay between eligibility criteria and choice of adjustment. FIG. 9A shows results of the parametric simulations where the least at risk patients are selected in the 50% inclusion. FIG. 9B shows results of the semi-synthetic simulation based on the HCC-TCGA dataset. The three levels of inclusion are based on eligibility criteria of past and ongoing trials.

FIG. 10 illustrates an example of a computer system, which may be used in conjuncture with the embodiments described herein.

### DETAILED DESCRIPTION

Systems and methods for covariate analysis is described. In some aspects, provided herein is a method of using the Cox-Snell R². In some embodiments, a prognostic score for a disease, e.g., cancer, obtained using deep-learning on histological slides can be used as a covariate to be adjusted using the Cox-Snell R².

In the following description, numerous specific details are set forth to provide thorough explanation of embodiments of the present invention. It will be apparent, however, to one skilled in the art, that embodiments of the present invention may be practiced without these specific details. In other instances, well-known components, structures, and techniques have not been shown in detail in order not to obscure the understanding of this description.

Reference in the specification to “one embodiment” or “some embodiments” means that a particular feature, structure, or characteristic described in connection with the embodiment can be included in at least one embodiment of the invention. The appearances of the phrase “in one embodiment” in various places in the specification does not necessarily all refer to the same embodiment. The term “exemplary” is used herein in the sense of “example,” rather than “ideal.” From this disclosure, it should be understood that the invention is not limited to the examples described herein.

For any methods described herein, the ordering of steps as presented, whether in the text or in an accompanying flow diagram, should not be taken to mean that those steps must be performed in the order presented, unless otherwise specified or required by context. Rather, the order of steps presents one embodiment of the methods provided, and in general such steps may alternatively be performed in a different order or simultaneously. The processes depicted in the figures that follow may be performed by processing logic that comprises hardware (e.g., circuitry, dedicated logic, etc.), software (such as is run on a general-purpose computer system or a dedicated machine), or a combination of both. Although the processes are described below in terms of some sequential operations, it should be appreciated that some of the operations described may be performed in different order. Moreover, some operations may be performed in parallel rather than sequentially.

Computing methods used for implementing the methods provided herein can include, for example, machine learning, artificial intelligence (AI), deep learning (DL), neural networks, classification and/or clustering algorithms, and regression algorithms.

The terms “server,” “client,” and “device” are intended to refer generally to data processing systems rather than specifically to a particular form factor for the server, client, and/or device.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to.” The term “including” does not necessarily imply that additional elements beyond those recited must be present.

The term “about” or “approximately” when referring to a number or a numerical range means that the number or numerical range referred to is an approximation within experimental variability (or within statistical experimental error), and, thus, the number or numerical range may vary from, for example, between 1% and 20% of the stated number or numerical range. In some aspects, “about” indicates a value within 20% of the stated value. In more preferred aspects, “about” indicates a value within 10% of the stated value. In even more preferred aspects, “about” indicates a value within 1% of the stated value.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in aspects of the present invention. Notwithstanding that the numerical ranges and parameters setting forth the broad scope of the invention are approximations, the numerical values set forth in the specific examples are reported as precisely as possible. Any numerical values; however, inherently contain certain errors necessarily resulting from error found in their respective measurements.

The term “at least” prior to a number or series of numbers is understood to include the number adjacent to the term “at least”, and all subsequent numbers or integers that could logically be included, as clear from context. When “at least” is present before a series of numbers or a range, it is understood that “at least” can modify each of the numbers in the series or range.

As used herein, “no more than” or “less than” is understood as the value adjacent to the phrase and logical lower values or integers, as logical from context, to zero (if negative values are not possible). When “no more than” is present before a series of numbers or a range, it is understood that “no more than” can modify each of the numbers in the series or range.

As used herein, “up to” as in “up to 10” is understood as up to and including 10, i.e., 0, 1, 2, 3, 4, 5, 6, 7, 8, 9, or 10, in the context of non-negative integers.

Where a range of values is provided, it is understood that each intervening value (e.g., to the tenth of the unit of the lower limit unless the context clearly dictates otherwise) between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

A “patient” refers to a subject who shows symptoms and/or complications of a disease or condition (e.g., cancer), is under the treatment of a clinician (e.g., an oncologist), has been diagnosed as having a disease or condition, and/or is at a risk of developing a disease or condition. The term “patient” includes human and veterinary subjects. Any reference to subjects in the present disclosure should be understood to include the possibility that the subject is a “patient” unless clearly dictated otherwise by context.

As used herein, “predict” or “predicting” refers to determining a likelihood that a disease, a condition, or an event (e.g., death of a cancer subject) happens in the future. In some embodiments, a model (e.g., DL model) can predict a likelihood of survival by one or more of the following measures of test accuracy:
an odds ratio greater than 1, preferably about 2 or more or about 0.5 or less, about 3 or more or about 0.33 or less, about 4 or more or about 0.25 or less, about 5 or more or about 0.2 or less, or about 10 or more or about 0.1 or less;
a specificity of greater than 0.5, preferably at least about 0.6, at least about 0.7, at least about 0.8, at least about 0.9, or at least about 0.95, with a corresponding sensitivity greater than 0.2, preferably at least about 0.3, at least about 0.4, at least about 0.5, at least about 0.6, at least about 0.7, at least about 0.8, at least about 0.9, or at least about 0.95;
a sensitivity of at least 0.5, preferably at least about 0.6, at least about 0.7, at least about 0.8, at least about 0.9, or at least about 0.95, with a corresponding sensitivity at least 0.2, preferably at least about 0.3, at least about 0.4, at least about 0.5, at least about 0.6, at least about 0.7, at least about 0.8, at least about 0.9, or at least about 0.95;
at least about 75% sensitivity, combined with at least about 75% specificity;
a positive likelihood ratio [calculated as sensitivity/(1−specificity)] of greater than 1, preferably at least about 2, at least about 3, at least about 4, at least about 5, at least about 10; or
a negative likelihood ratio [calculated as (1−sensitivity)/specificity] of less than 1, preferably about 0.5 or less, about 0.33 or less, about 0.25 or less, or about 0.1 or less.

As used herein, “risk score” refers to a likelihood of a certain event, e.g., a disease, relapse, is going to happen in the future. In some embodiments, the risk score represents the likelihood that the patient will experience a relapse following treatment. In some embodiments, the risk score is expressed as a classification. In other embodiments, the risk score is expressed as a continuous range. In one embodiment, the risk score represents overall survival, i.e., the duration of time from a cancer diagnosis to the death of a subject.

As used herein, a “subject” is an animal, such as a mammal, including a primate (such as a human, a monkey, and a chimpanzee) or a non-primate (such as a cow, a pig, and a horse) that benefits from the methods according to the present disclosure. In some aspects of the invention, the subject is a human, such as a human diagnosed with cancer. The subject may be a female human. The subject may be a male human. In some aspects, the subject is an adult subject.

### I. Overview of the Invention

Adjustment on covariates (e.g., prognostic covariates) allows for improved precision and increased statistical power for treatment effect estimation in studies, e.g., randomized controlled trials (RCTs). However, sample size calculations under covariate adjustment typically require knowledge of the magnitude of adjustment covariates, which is usually not available at the commencement of the trial, especially with novel endpoints and/or indications. It is therefore common to start with a preliminary calculated sample size based on the sparse information available in the planning phase and to re-estimate the value of the adjustment covariates (and with it the sample size) when a portion of the planned number of patients have completed the study (Friede & Kieser 2011 Pharmaceut. Statist. 10:8-13). In this regard, it is important that the sample size reestimation is performed such that all persons involved in the study remain blinded to the treatment group allocation and that the procedure does not inflate the Type I error rate.

The induced reduction in sample size, driven by reduction in number of events to achieve a targeted statistical power, can be estimated based on a known correlation between the adjustment covariate and the outcome when the outcome is continuous. In contrast, for a time-to-event outcome, e.g., survival data, there is not a unique definition for the proportion of variation explained by a covariate. There is no clear way to take the degree of association between a covariate and the outcome into account when computing the sample size before starting a trial (e.g., pre-trial sample size designing), or during the trial when a portion of the data becomes available (e.g., blinded sample size reestimation).

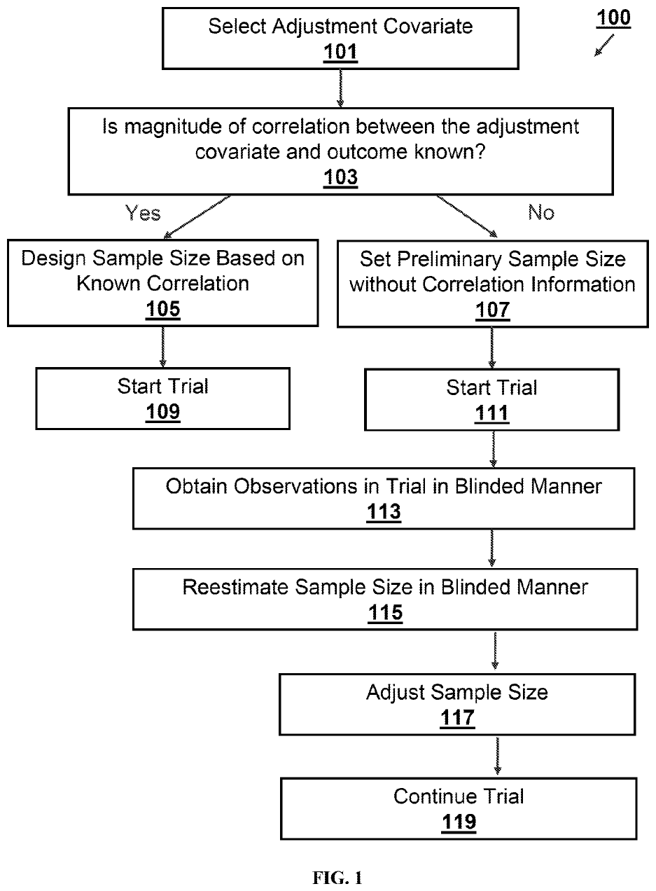

Accordingly, the present disclosure provides methods for designing (e.g., reducing) sample size in a trial based on known correlation of the adjustment covariate and outcome, both in the continuous and time-to-event settings. The present disclosure further provides methods for reestimating and readjusting sample size while maintaining the blindness of the trial, e.g., conducting blinded sample size reestimation, based on data that became available during the trial, including the adjustment covariate and outcome data, both in the continuous and time-to-event settings. These methods are outlined for instance in FIGS. 1-3.

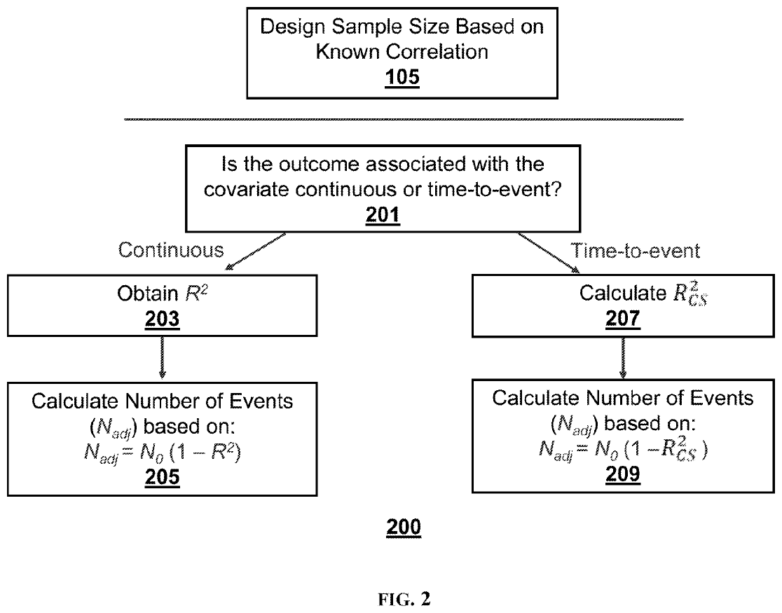

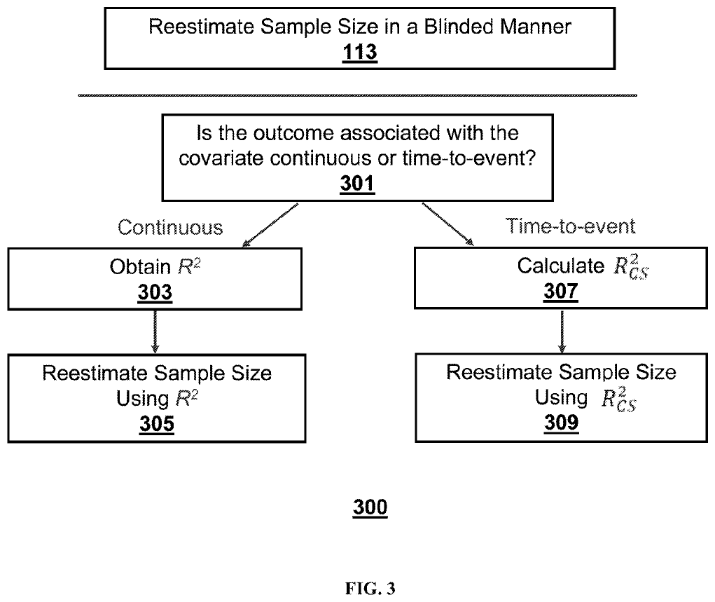

Specifically, the parametric simulations in the present disclosure provide that predictive performance (C-index) and outcome incidence in the trial are the two main determinants of the reduction in sample size. Based on analysis of several proposed generalizations of the explained proportion of variability, the present disclosure identifies that the Cox-Snell R_{CS}² best fits the observed sample size reduction in the covariate adjustment settings with a time-to event outcome. The present disclosure therefore provides methods for designing a study by computing sample size in a covariate adjusted analysis using the Cox-Snell R_{CS}² in a time-to-event outcome setting (FIG. 2). The present disclosure also provides methods for reestimating a sample size in a blinded manner using the Cox-Snell R s in a covariate adjusted analysis a time-to-event outcome (FIG. 3).

The methods disclosed herein can incorporate novel sources of prognostic signals, e.g., prognostic scores obtained by deep learning, as adjustment covariates to compute the adjusted (e.g., reduced) sample size. Using the publicly available clinical trial information on hepatocellular carcinoma and mesothelioma, the present disclosure provides verification that such covariate adjustment methods using deep learning prognostic covariates can improve the statistical power achieved based on the same sample size, and can reduce sample size needed to achieve the same statistical power.

Further provided herein are methods for achieving a targeted statistical power using less strict eligibility criteria in a trial by using covariate adjustment that can compensate for the increased heterogeneity that comes with less restrictive inclusion criteria. Thus, systematic adjustment on prognostic covariates according to the present disclosure can lead to reductions in sample size needed for a targeted power as well as efficient and inclusive clinical trials (e.g., relaxation of eligibility criteria) especially in disease settings where the outcome incidence is high, e.g., in metastatic cancer.

In one embodiment, reducing the sample size used for a randomized clinical trial (or reestimating the sample size in a blinded manner) improves the functioning of a device that is used to compile the results of the randomized clinical trial. In this embodiment, reducing randomized clinical trial sample size means that a smaller number of participants are needed to achieve the same statistical power in the randomized clinical trial. In addition, by having a smaller number of participants, the randomized clinical trial compiles a smaller amount of data. This improves the efficiency of the device that is compiling, analyzing, storing, and/or otherwise processing this data as there is a smaller amount of data resulting from a randomized clinical trial with a smaller sample size. This improves on the efficiency of the device by reducing the computing and storing load on that device.

### II. Parametric Simulations for Covariate Adjustment and Sample Size Reduction

The Fleiss formula below depicts the relationship between the number of events required for a given statistical power for the unadjusted analysis (denoted N_{original}) and for the adjusted analysis (denoted N_{adjusted}) with respect to the correlation (denoted r) between the outcome and the adjustment covariate in the continuous outcome setting:

$${N}_{adjusted} = {N}_{original} ( 1 - {R}^{2} ) .$$

Parametric simulations are performed to compute the observed sample size reduction R_{obs}² as a function of a single adjustment covariate's C-index and incidence of the outcome in the continuous outcome as well as time-to-event outcome settings. As used herein, the “C-index” is a measure of the model's prediction ability. The C-index is controlled via a covariate coefficient. As used herein, the “outcome incidence” refers to number of events/sample size. In sample size cases of survival data, the outcome incidence can be estimated with a Kaplan-Meier curve. Other parameters of interest are the size of the treatment effect, the Weibull shape of the baseline hazard function, and the drop-out rate. The simulations are made under the proportional hazard assumption as in the Cox model.

Survival times are generated following the Weibull distribution with shape w and scale depending on the treatment hazard ratio hr, and on a standard Gaussian covariate x. Censored times are drawn from an exponential distribution with a specified drop-out rate d. Denoting z the treatment allocation variable, this generative model can be formally summarized as follows for patient i:

$$\{ \begin{matrix}{h}_{i} ( z ) = {hr}^{z} exp ( κ + θ {x}_{i} ) , \\ {T}_{i} ( z ) \sim 𝒲 ( {{h}_{i} ( z )}^{- 1 / w} , w ) , \\ {T}_{i}^{drop} \sim ℰ ( d )\end{matrix} .$$

All patients remaining at risk at 5 years are censored at that time. The treatment allocation is independent from the covariate and the arms of the trial are balanced. For each set of input parameters, the auxiliary parameters and are numerically optimized κ and θ are numerically optimized to reach the aimed outcome incidence λ, at 5 years in the placebo arm, and the C-index C evaluated on the whole trial population.

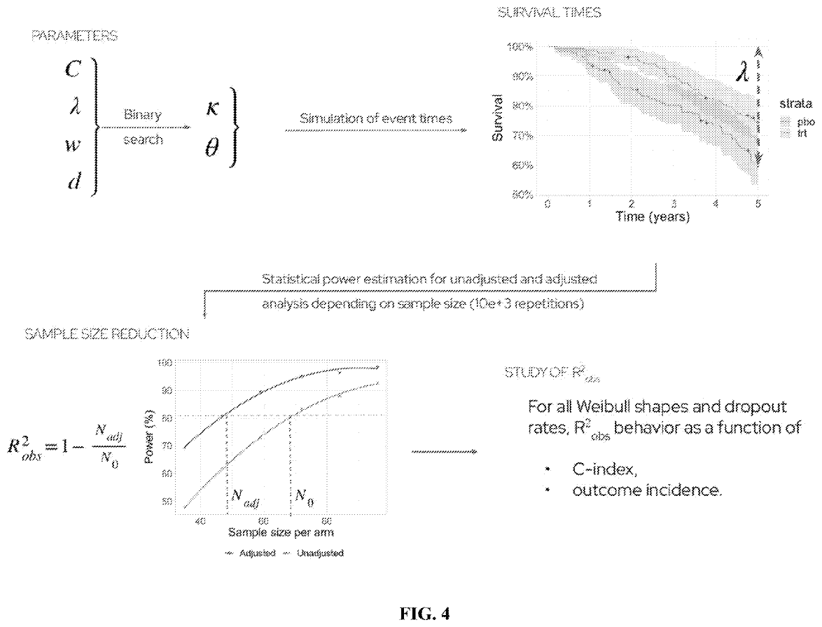

FIG. 4 illustrates the process for the computation of R_{obs}². Once survival times are simulated, the statistical power for the unadjusted model and the model adjusted for the X covariate is estimated on a grid of sample sizes as the percentage of detected treatment effect on 10e+3 resamples (Heo et al. 1998 Mech Ageing Dev 102:45-53). The resulting power curves give the number of events N_{adjusted} and N_{original} required to reach a power of 80% for both models, from which R_{obs}² is deduced. These simulations explore a wide range of parameter values as shown in Table 1, allowing for an extensive study of R_{obs}² behavior as a function of λ and C in different settings of proportional hazards.

TABLE 1
Description of simulation parameters

| Quantity | Notation | Values |
|---|---|---|
| Outcome | λ | {0.1, 0.2, 0.3, 0.4, 0.5, 0.6, 0.7, 0.8, 0.9} |
| incidence | | |
| C-index | C | {0.55, 0.65, 0.75, 0.85} |
| Weibull shape | w | {0.5, 1, 1.5} |
| Drop-out rate | d | {0.01, 0.1} |
| Treatment hazard | hr | {0.4, 0.7} |
| ratio | | |

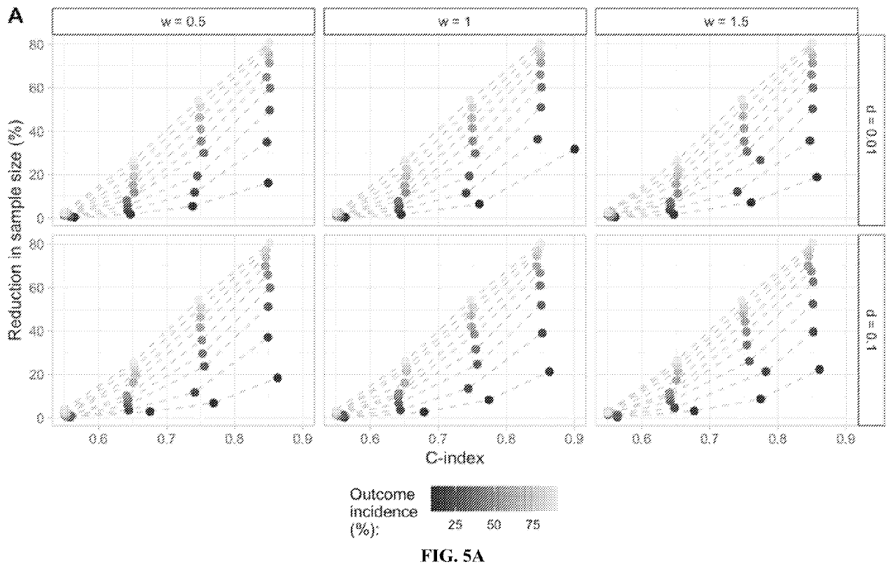

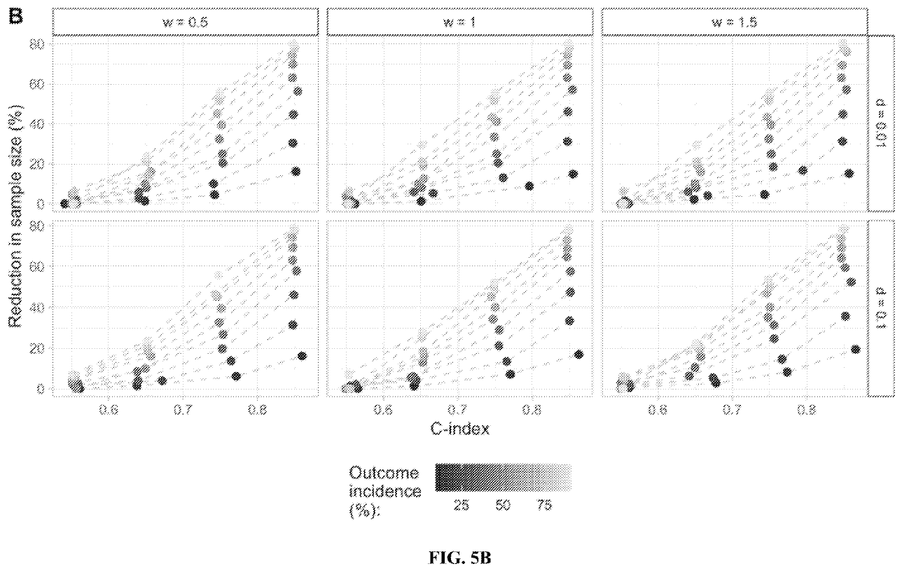

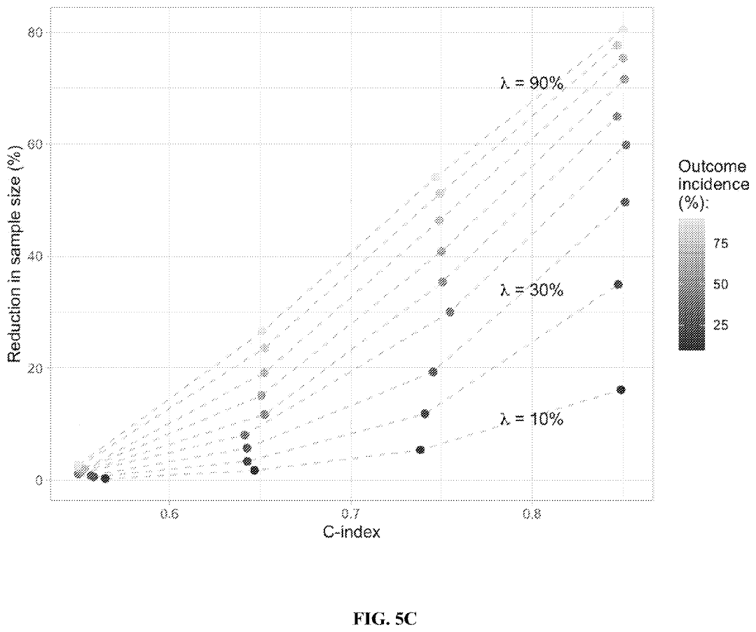

The parametric simulations show that the sample size reduction obtained with covariate adjustment increases both with the model's C-index and the outcome incidence. FIGS. 5A-5C show behavior of R_{obs}² as a function of c-index, outcome incidence, treatment effect, Weibull shape, and drop-out rate (A, C: hr=0.7, B: hr=0.4). The influence of the outcome incidence grows with the C-index. For instance, in FIG. 5C, which is the same chart as the top left panel of FIG. 5A (hr=0.7, w=0.5, d=0.01), when an outcome incidence of k=10% and λ=90% are compared, there is a difference of 5 percentage points in reduction of sample size for a C-index of 0.55, and of 60 percentage points for a C-index of 0.85. Further, FIGS. 5A-5B show that the relationship of sample size reduction with C-index and outcome incidence does not depend on the size of the treatment effect, the Weibull shape parameter or the drop-out rate. The values chosen for the drop-out rate, d=0.01 or d=0.1, result in a median of censored patients before the end of follow-up of 3.7% (range 0.7%-4.8%) and 28.1% (range 5.4%-38.4%), respectively.

The parametric simulations provided herein demonstrate that the main determinants of the reduction in sample size needed for a given power are the C-index of the adjustment covariates and outcome incidence in the trial. Other parameters such as Weibull shape, drop-out rate, or effect size do not impact the precision gains obtained by covariate adjustment, consistent with previous report (Hernandez et al. 2006 Ann Epidemiol 16:41-8). The present disclosure uniquely identifies the strong dependence of sample size reduction on outcome incidence in simulation settings of a finite time horizon (i.e. follow-up stops at 5 years).

Identification of the key parameters (e.g., outcome incidence, C-index) that determine sample size reduction by covariate adjustment helps prioritize the indications where more attention should be given to covariate adjustment. For example, in diseases with high prognostic outcome incidence, e.g., metastatic cancers, aggressive cancers, e.g., mesothelioma (Kerr et al. 2017 Clin Trials 14:629-38), covariate adjustment will be impactful, with every additional point of C-index translating to notable gains in precision. In diseases with low prognostic outcome incidence, e.g., secondary cardiovascular prevention, prognostic signal can be used to perform prognostic enrichment additionally or alternatively to covariate adjustment.

### III. Designing Sample Size by Adjusting for Covariate Based on Time-to-Event Data

Covariate adjustment increases statistical power and leads to more precise estimates of treatment effect. FIG. 1 shows an exemplary flow diagram of designing the sample size of a trial, e.g., a randomized controlled trial (RCT) according to the methods of the present disclosure. Process 100 begins with selecting an adjustment covariate at block 101. As used herein, an “adjustment covariate” can be any variable, other than the treatment allocation that is tested in the trial, which is correlated with the outcome. An adjustment covariate can be associated with a continuous outcome. Alternatively, an adjustment covariate can be associated with a non-continuous (e.g., time-to-event) outcome. Sample size calculations under covariate adjustment can be performed based on the magnitude of adjustment covariates with regard to the outcome. Accordingly, at block 103, process 100 investigates whether a magnitude of correlation between the adjustment covariate and outcome is known. If yes, process 100 proceeds to block 105, where it designs sample size based on known correlation. Block 105 is further detailed in FIG. 2. Based on the sample size designed at block 105, process 100 proceeds to start a trial at block 109.

FIG. 2 shows an exemplary flow diagram of designing the sample size of a trial based on a known correlation between the adjustment covariate and the outcome. Block 105 is depicted as process 200. Process 200 begins by investigating whether the outcome associated with the adjustment covariate is continuous or time-to-event, at 201. For a continuous outcome, there is a formula connecting the number of events required for a given statistical power for the unadjusted (denoted N_{original}) and for the adjusted analysis (denoted N_{adjusted}) in terms of the correlation between the outcome and the adjustment covariate (denoted R):

$${N}_{adjusted} = {N}_{original} ( 1 - {R}^{2} ) .$$

For instance, a correlation of 0.5 between a baseline covariate and the outcome translates to sample size requirements for the adjusted analysis reduced by 25% compared to the unadjusted analysis. This formula can be referred to as the Fleiss formula. Process 200 continues to obtain R² at 203, and to design the number of events N_{adjusted} based on the Fleiss formula above, at 205.

In contrast, if the outcome associated with the adjustment covariate is a time-to-event outcome at 201, e.g., survival data, there has not been a unique definition for the proportion of variation explained by a covariate. As provided herein in the parametric simulations, observed reduction of sample size (R_{obs}²) increases with an increase in predictive power of prognostic variables (C-index) and/or an increase in the incidence of the outcome (λ), with the effect of X greater for medium C-index values (e.g., between 0.7 and 0.8) than C-index values in other ranges. However, there has been no clear way to take the association between a covariate and the outcome into account when computing sample size in time-to-event outcome settings. The present disclosure provides novel methods for designing a study by computing sample size in a covariate adjusted analysis using the Cox-Snell R_{CS}² in the Fleiss formula. That is, if the outcome associated with the adjustment covariate is a time-to-event at 201, process 200 continues to calculate R_{CS}² at 207, and to design the number of events N_{adjusted} using R_{CS}² instead of R² in the Fleiss formula, at 209.

To arrive at the methods described herein, e.g., those exemplified in FIG. 2, different ways of generalizing R² were implemented and whether any means would generalize the Fleiss formula for the time-to-event setting was investigated using parametric simulations with a finite time horizon.

Among several categories of proposed measures generalizing the classical R² to time-to-event data, explained variation (EV) and explained randomness (ER) measures were examined. Explained variation (EV) measures are extensions of the classical measure of proportion of variance explained by a set of covariates used in linear regression. Explained randomness (ER) measures, on the other hand, build on the concept of entropy and compare the quantity of information contained in models with and without the covariates of interest. In the present disclosure, three EV measures [R_{D}² (Royston & Sauerbrei 2004 Stat Med 23:723-48), R_{PM}² (Kent & O'Quigley 1988 Biometrika 75:525-534), and R_{R} (Royston 2006 Stata J Promot Commun Stat Stata 6:83-96)] and five ER measures [Rᵢ² (Royston & Sauerbrei 2004 Stat Med 23:723-48), ρₖ² (O'Quigley et al. 2005 Stat Med 24:479-89), ρ_{WA}² (Royston & Sauerbrei 2004 Stat Med 23:723-48), ρ_{XO}² (Ronghui & O'Quigley 1999 J Nonparametric Stat 12:83-107) and R_{CS}² (Cox et al. 1989 Analysis of binary data. 2. ed., 1. CRC Press reprint. Boca Raton, Fla., Chapman & Hall)]. As used herein, the term “proposed R² measures” refers collectively to potential R² measures investigated, including R_{D}², R_{PM}², and R_{R}², Rᵢ², ρₖ², ρ_{WA}², ρ_{XO}², and R_{CS}². For instance, denoting l₀ and l₁ the log-likelihoods of a base model and a model adjusting for additional covariates, provided is:

$${R}_{CS}^{2} = 1 - exp [ - \frac{2}{n} ( {l}_{1} - {l}_{0} ) ] .$$

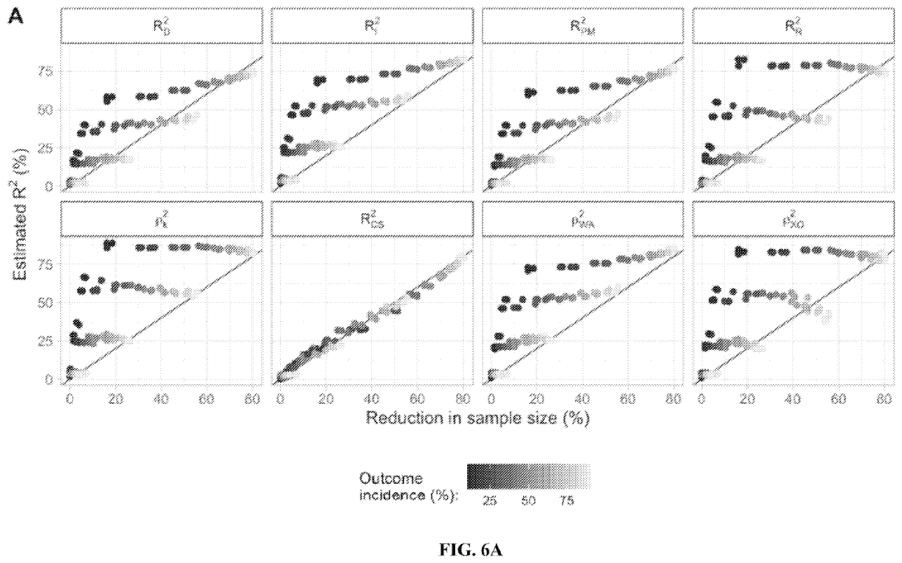

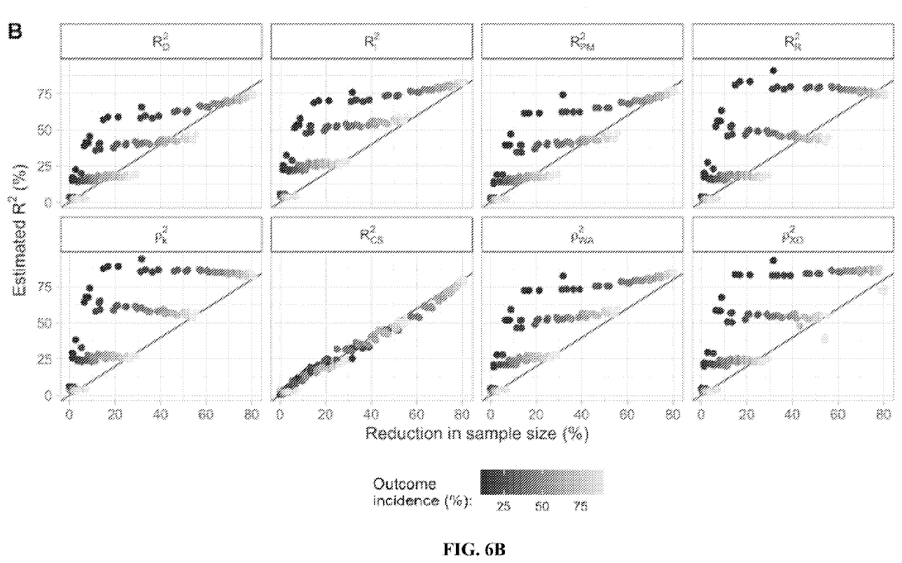

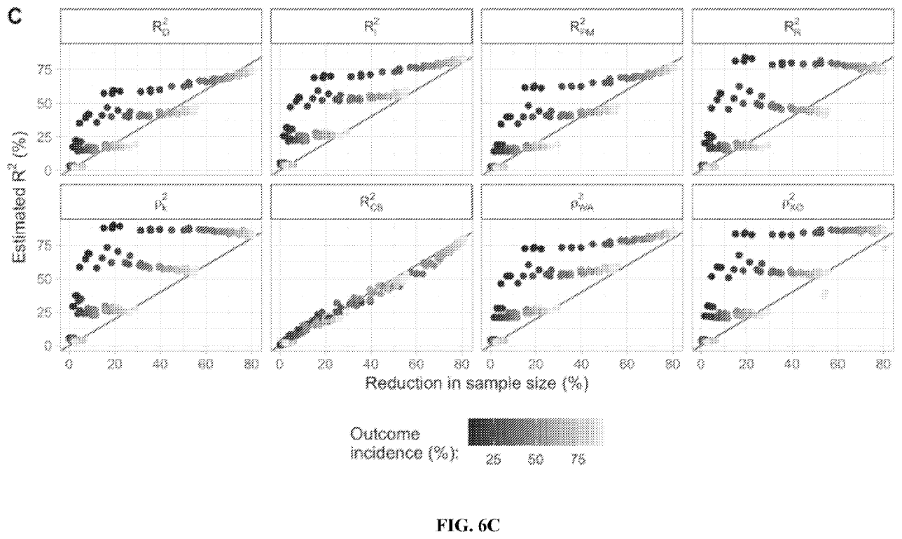

FIGS. 6A-6C gather all the R² measures values and compare them to R_{obs}² values for each Weibull shape. Strikingly, only two out of eight measures (R_{CS}² and ρ_{XO}²) actually show values increasing with the outcome incidence. Others only increase with the C-index value and remain overall constant with respect to the outcome incidence, creating steps. This is due to the fact that most measures were developed with a robustness to censoring, which is directly linked to the outcome incidence in a finite time horizon setting. The Cox & Snell R_{CS}² outperforms other proposed R² measures and best captures the observed sample size reduction in all our simulations: the median absolute error of R_{CS}² is 2.1% (first and third quartiles are 0.8% and 3.9%, respectively).

Accordingly, provided herein is a metric that generalizes the Fleiss formula to non-continuous data (e.g., time-to-event) setting: the Cox-Snell R_{CS}². Having such a metric is of practical importance as it can help design clinical trials without the help of simulations. Provided herein is a method for designing a randomized controlled trial (RCT), said method comprising calculating a number of events required to obtain a statistical power based on a formula:

$${N}_{adjusted} = {N}_{original} ( 1 - {R}_{C S}^{2} )$$

wherein the N_{original} is a number of events required to obtain the statistical power without covariate adjustment, and the N_{adjusted} is a number of events required to obtain the statistical power with covariate adjustment. In some embodiments, the methods of the present disclosure enable designing clinical trials without using simulations. The exemplary method is depicted in FIG. 2 at 201, 207, and 209. In some embodiments, the RCT is conducted based on the calculated number of events.

### IV. Blinded Sample Size Reestimation for Covariate Adjustment Based on Time-to-Event Data

The metric and methods of the present disclosure can also be useful in the case of a blinded sample size reestimation during a trial when there is uncertainty on the predictive performance of adjustment covariates at the start of the trial. Sample size calculations under covariate adjustment typically require knowledge of the magnitude of correlation between adjustment covariates and the clinical outcome. However, when planning the sample size of a trial it can be difficult to obtain good estimates of this correlation from previous studies especially with novel endpoints and/or indications. An exemplary workflow of blinded sample size reestimation in a trial, e.g., an RCT, is depicted in FIG. 1. At block 103, process 100 investigates as to whether magnitude of correlation between the adjustment covariate and outcome is known. If no, process 100 proceeds to block 107, where it sets preliminary sample size based on known information without the correlation information. In some embodiments, based on an initial guess of the value of the covariates, a provisional sample size is calculated. Based on the sample size designed based on the preliminary sample size set without the correlation information at block 107, process 100 continues to block 111 and starts the trial.

As the trial progresses, data is obtained from subjects in a blinded manner i.e., without being known to the investigators whether the subject belongs to the treatment group or the control group. Process 100 continues to block 113, where it obtains observations in the trial in a blinded manner.

When the observations are available for a prespecified portion of this sample size, the correlation between adjustment covariates and the outcome are re-estimated and, if necessary, the sample size is adapted accordingly. From a regulatory perspective it is important that the sample size recalculation is performed such that all persons involved in the study remain blinded to the treatment group allocation and that the procedure does not inflate the Type I error rate (Friede & Kieser 2011 Pharmaceut. Statist. 10:8-13). At 115, process 100 conducts blinded sample size reestimation based on the observations obtained in the ongoing trial, including data regarding the adjustment covariates and the outcome. Block 115 is further detailed in FIG. 3.

FIG. 3 shows an exemplary flow diagram of blinded sample size reestimation e.g., during a trial. Block 113 is depicted as process 300. Process 300 begins by investigating whether the outcome associated with the adjustment covariate is continuous or time-to-event, at 301. In continuous outcome settings, process 300 continues to block 303, where the correlation (denoted R²) between the adjustment covariate and the outcome is obtained based on the data obtained during the trial. Process 300 continues to block 305, where it reestimates the sample size using R². The sample size can be reestimated according to certain procedure reported for a blinded sample size reestimation, for instance by Friede & Kieser 2011 Pharmaceut. Statist. 10:8-13 and Zimmermann et al. 2020 Univ. Kentucky, Statistics Faculty Publications 28, each of which is hereby incorporated by reference in its entirety.

For instance, the reestimated number of events can be obtained by the following formula:

$${N}_{adjusted} = {N}_{original} ( 1 - {R}^{2} ) ;$$

wherein the N_{original} is the original number of events required to obtain the statistical power without covariate adjustment, the N_{adjusted} is a reestimated number of events required to obtain the statistical power, and the R is the correlation between the outcome and the adjustment covariate based on the blinded RCT data. The RCT can be further conducted using the blinded sample size reestimation. In some embodiments, the N_{original} is evaluated based on a formula:

N_{original}=(z_{β}+z_{1-α})²/(P₁P₂ log² (hr)), wherein
the N_{original} is an estimated number of events required to obtain the statistical power based on the Schoenfeld formula (Schoenfeld, 1983 Biometrics, 39:2:499-503),
the α is a type I error level,
the β is a type II error level,
the P₁ and the P₂ are the proportion of the trial sample included in the treatment and control arm respectively (e.g. both are equal to ½ if the treatment allocation is balanced),
the hr is a stipulated hazard ratio, and
the zₚ is a p-quantile of the standard normal distribution.

Additionally or alternatively, the adjusted number of events (denoted NA) can be obtained by a basic approximate formula:

$${N}_{A} = \frac{{( γ + 1 )}^{2}}{γ} \frac{{( {z}_{1 - α / 2} + {z}_{1 - β} )}^{2} {σ}_{Y}^{2} ( 1 - {R}^{2} )}{{Δ}^{2}} ,$$

wherein
the N_{A} and the N_{GS} are each a reestimated number of events required to obtain the statistical power,
the α is a type I error level,
the β is a type II error level,
the

$$γ = \frac{{n}_{2}}{{n}_{1}}$$

is an allocation ratio, wherein n₁ and n₂ are sample sizes of group 1 and 2, respectively,
the δ_{Y}² is a variance of outcome,
the Δ is a stipulated difference of adjusted means, and
the zₚ is a p-quantile of the standard normal distribution.
   Alternatively or additionally, the adjusted number of events (denoted N_{GS}, N_{DF}, or N_{GS,DF}) can be obtained based on the basic approximate formula with a Guenther-Schouten-like adjustment (GS):

$${N}_{GS} = {N}_{A} + \frac{{z}_{1 - α / 2}^{2}}{2} ;$$

or
the basic approximate formula with a degrees-of-freedom adjustment (DF):

$${N}_{DF} = {N}_{A} \frac{{N}_{A} - 2}{{N}_{A} - 2 - c} ;$$

or
the basic approximate formula with a combined Guenther-Schouten and degrees-of-freedom adjustment (GS+DF):

$${N}_{GS , DF} = {N}_{DF} + \frac{{z}_{1 - α / 2}^{2}}{2} .$$

In contrast, if outcome associated with the adjustment covariate is a time-to-event outcome, e.g., survival data, there has not been a unique definition for the proportion of variation explained by a covariate. There has been no clear way to conduct blinded sample size reestimation in time-to-event outcome settings when there is uncertainty on the magnitude of association between a prognostic covariate and the outcome. The present disclosure provides novel methods for conducting blinded sample size reestimation using the Cox-Snell R_{CS}². That is, if the outcome associated with the adjustment covariate is not continuous at block 301, process 300 continues to calculate R_{CS}² at block 307. R_{CS}² can be calculated based on the following formula in the interim data:

$${R}_{C S}^{2} = 1 - exp [ - \frac{2}{n} ( {l}_{1} - {l}_{0} ) ] ,$$

wherein
the R_{CS}² is the Cox-Snell R²,
the n is a number of participants,
the l₀ is a log-likelihood of a model without covariate adjustment, and
the l₁ is a log-likelihood of a model with covariate adjustment.

The process 300 continues to block 309, where it conducts blinded sample size reestimation using R_{CS}². The number of events can be evaluated based on the following formula:

$${N}_{adjusted} = {N}_{original} ( 1 - {R}_{CS}^{2} ) ;$$

wherein the N_{adjusted} is a reestimated number of events required to obtain the statistical power. The RCT can be further conducted using the blinded sample size reestimation.

In some embodiments, the N_{original} is evaluated based on a formula:

N_{original}=(z_{β}+z_{1-α})²/(P₁P₂ log² (hr), wherein
the N_{original} is an estimated number of events required to obtain the statistical power based on the Schoenfeld formula,
the α is a type I error level,
the β is a type II error level,
the P₁ and the P₂ are the proportion of the trial sample included in the treatment and control arm respectively (e.g. both are equal to ½ if the treatment allocation is balanced),
the hr is a stipulated hazard ratio, and
the zₚ is a p-quantile of the standard normal distribution.

At block 117, process 100 adjusts the sample size based on the results of blinded sample size reestimation at block 115. Process 100 then continues the trial at block 119 based on the adjusted sample size at block 117.

### V. Covariate Adjustment Based on Deep Learning Covariates

Provided herein are methods of increasing statistical power of studies, e.g., clinical trials, and/or reducing number of events required for a targeted power by incorporating novel sources of prognostic signal obtained using deep learning prognostic models as adjustment covariates. The deep learning prognostic models can be based on histopathological slides of cancer patients. In some embodiments, covariate adjustment is performed based on deep learning covariates. Histological slides are already used to obtain covariates used for adjustment but covariates are obtained by anatomopathologists to determine histological subtypes (e.g., MAPS trial). With the deep learning technology, the numerized slides are processed automatically with a deep learning model in addition to the more traditional clinical evaluation of subtypes.

### Deep Learning Model-Based Prognostic Prediction

Provided herein are methods for predicting patient outcome based on deep learning of histological slides for use in the context of covariate adjustment. Prediction of survival based on deep learning on histological slides can be performed using deep learning models described for instance in Saillard et al. 2020 Hepatology 72:6; 2000-2013 (HCCnet) and Courtiol et al. 2019 Nat Medicine 25:10; 1519-1525 (MesoNet), each of which is hereby incorporated by reference in its entirety.

Histology is the field of study relating to the microscopic features of biological specimens. Histopathology refers to the microscopic examination of specimens, e.g., tissues, obtained or otherwise derived from a subject, e.g., a patient, in order to assess a disease state. Histopathology specimens generally result from processing the specimen, e.g., tissue, in a manner that affixes the specimen, or a portion thereof, to a microscope slide. For example, thin sections of a tissue specimen may be obtained using a microtome or other suitable device, and the thin sections can be affixed to a slide. To assist in the visualization of the specimen, the specimen may optionally be further processed, for example, by applying a stain. Many stains for visualizing cells and tissues have been developed. These include, without limitation, Haemotoxylin and Eosin (H&E), methylene blue, Masson's trichome, Congo red, Oil Red O, and safranin. H&E is routinely used by pathologists to aid in visualizing cells within a tissue specimen. Hematoxylin stains the nuclei of cells blue, and eosin stains the cytoplasm and extracellular matrix pink. A pathologist visually inspecting an H&E stained slide can use this information to assess the morphological features of the tissue.

However, H&E stained slides generally contain insufficient information to assess the presence or absence of particular biomarkers by visual inspection. Visualization of specific biomarkers (e.g., protein or RNA biomarkers) can be achieved with additional staining techniques which depend on the use of labeled detection reagents that specifically bind to a marker of interest, e.g., immunofluorescence, immunohistochemistry, in situ hybridization, etc. Such techniques are useful for determining the expression of individual genes or proteins, but are not practical for assessing complex expression patterns involving a large number of biomarkers. Global expression profiling can be achieved by way of genomic and proteomic methods using separate samples derived from the same tissue source as the specimen used for histopathological analysis. Notwithstanding, such methods are costly and time consuming, requiring the use of specialized equipment and reagents, and do not provide any information correlating biomarker expression to particular regions within the tissue specimen, e.g., particular regions within the H&E stained image.

As used herein, the term “digital image” refers to an electronic image represented by a collection of pixels which can be viewed, processed and/or analyzed by a computer. In some aspects of the present disclosure, digital images of histology slides, e.g., H&E stained slides, allow computational assessment of tissue specimens, in addition to or alternatively to visual inspection by a pathologist. In some embodiments, a digital image can be acquired by means of a digital camera or other optical device capable of capturing digital images from a slide, or portion thereof. In other embodiments, a digital image can be acquired by means of scanning a non-electronic image of a slide, or portion thereof. In some embodiments, the digital image used in the applications provided herein is a whole slide image. As used herein, the term “whole slide image (WSI),” refers to an image that includes all or nearly all portions of a tissue section, e.g., a tissue section present on a histology slide. In some embodiments, a WSI includes an image of an entire slide. In other embodiments, the digital image used in the applications provided herein is a selected portion of a tissue section, e.g., a tissue section present on a histology slide. In some embodiments, a digital image is acquired after a tissue section has been treated with a stain, e.g., H&E.

In some aspects, the computer-implemented methods of predicting a likelihood of certain prognosis, e.g., relapse, overall survival, or disease free survival comprises the following:
accessing a digital histology image of a histology section obtained from the subject;
extracting a plurality of feature vectors of the histology image by applying a first convolutional neural network, wherein each of the features of the plurality of feature vectors represents local descriptors of the histology image;
classifying the histology image using at least the plurality of feature vectors and a classification model, wherein the classification model is trained using a training set of known histology images and known prognosis information; and
determining the likelihood of prognosis of the subject based on at least the classification of the histology image.

In some aspects, the computer-implemented methods of predicting a likelihood of certain prognosis, e.g., relapse, overall survival, or disease free survival comprises the following:
obtaining a digital image of a histology section from the subject;
dividing the digital image into a set of tiles;
extracting a plurality of feature vectors from the set of tiles, or a subset thereof;
computing an artificial intelligence (AI) risk score based on the histology image using a machine learning model, the machine learning model having been trained by processing a plurality of training images to predict a likelihood of certain prognosis, e.g., relapse, overall survival, or disease free survival,
wherein the Al risk score represents the likelihood of prognosis of the subject.

In some embodiments, the methods can further comprise:
obtaining clinical attributes derived from the subject;
computing a clinical risk score using a clinical model, the clinical model trained using one or more subject attribute; and
computing a final risk score for the subject from the machine learning risk score and the clinical risk score, wherein the final risk score represents the likelihood of prognosis of the subject.

In some embodiments, the digital image of the methods is a whole slide image (WSI). In some embodiments, the histologic section of the disease (e.g., cancer) sample has been stained with a dye to visualize the underlying tissue structure, for example, with hematoxylin and eosin (H&E). Other common stains that can be used to visualize tissue structures in the input image include, for example, Masson's trichome stain, Periodic Acid Schiff stain, Prussian Blue stain, Gomori trichome stain, Alcian Blue stain, or Ziehl Neelsen stain.

The machine learning model can be a self-supervised machine learning model using histology data (such as whole slide images) by extracting features from tiles of the whole slide image. In one embodiment, feature extractor is trained on in-domain histology tiles, without annotations. In one embodiment, to apply a self-supervised framework on histology data, tiles are concatenated from all WSIs extracted at the tiling step to form a training dataset. The feature extractor is then trained with MoCo v2 on this set of unlabeled tile images. Initially, a set of tiles can be divided into two batches of tiles. A first batch of tiles and the second batch of tiles can be modified by, for example, adding 90° rotations and vertical flips, and also performing color augmentations. Since histology tiles contain the same information regardless of their orientation, rotations are good augmentations to perform. Because histology tiles are images that contain cells or tissue, and are not orientation dependent, such tiles can be viewed properly without regard for rotations, horizontal flip, etc. Thus, rotating the images provides a valuable augmentation without losing any important characteristics of the image. By applying the batches of tiles to their respective feature extractors, tile embeddings can be generated.

In one embodiment, the tile embeddings are the output of the feature extractors, and serve as a signature for each tile that includes semantic information for each tile. In other words, a tile embedding is a representation of a tile that contains semantic information about that tile.

In some embodiments, a self-supervised learning algorithm uses contrastive loss to shape the tile embeddings such that different augmented views of the same image are close together, or have similar tile embeddings. In other words, contrastive loss can compare the two tile embeddings, and based on that comparison the first feature extractor can be adjusted so that its tile embedding is similar to the tile embedding of the second feature extractor. Gradients are back-propagated through the first feature extractor. In some embodiments, the second feature extractor's weights are updated with an exponential moving average (EMA) of the first extractor's weights. The use of the EMA can avoid overfitting, in some embodiments. Thus, the output of this system is a trained feature extractor, which has been trained using in-domain histology tiles so that tile embeddings of various augmentations of the same image are similar. This type of specifically trained feature extractor can provide significant improvements in downstream performance, as discussed below. In some embodiments, the trained feature extractor can be achieved after training for a certain number of epochs. In some embodiments, training is performed until precision is at or near 1 (or 100%), until the AUC is at or near 1 (or 100%), or until the loss is near zero. In some embodiments, during training of a feature extractor with contrastive loss one may not have access to an abundance of helpful metrics. Thus, one can monitor one of the available metrics of downstream tasks, like AUC, to see how the feature extractor is performing. In one example, a feature extractor that is trained at a certain epoch can be used to train a downstream weakly supervised task in order to evaluate performance. If additional training could result in improved downstream performance, such additional training may be warranted.

In some embodiments, the second feature extractor can be optional, and a single feature extractor can be used to generate the tile embeddings from the two batches of tiles. In such an embodiment, one feature extractor is used to generate the tile embeddings from the two batches of tiles and contrastive loss is used, as described above, to compare the two tile embeddings, and adjust the first feature extractor so that the first tile embedding is similar to the second tile embedding.

In some embodiments, a machine learning algorithm extracts a plurality of feature vectors from the digital image and the extracting a plurality of feature vectors is performed using a first convolutional neural network, e.g., a ResNet50 neural network.

In some embodiments, the computer-implemented method further comprises removing background segments from the image. In some embodiments, removing background segments from the image is performed using a second convolutional neural network. In some embodiments, the second convolutional neural network is a semantic segmentation deep learning network.

In some embodiments, the computer-implemented method further comprises selecting a subset of tiles for application to the machine learning model. In some embodiments, the subset of tiles is selected by random sampling.

In some embodiments, the machine learning model is trained using a plurality of training images and the plurality of training images comprise digital images of histology sections of disease, e.g., cancer samples derived from a plurality of control subjects having said disease, e.g., cancer. In some embodiments, the plurality of training images comprise images that lack local annotations. In some further embodiments, the plurality of training images comprise images associated with one or more global label(s) indicative of one or more disease feature(s) of the control patient from whom the sample is derived.

In one embodiment of self-supervised learning on histology images to train a feature extractor, the process begins with receiving a training set of histology images. In some embodiments, each image in the training set of images is an annotation-free whole slide image. The process then continues with tiling and augmenting the training set of images into sets of tiles. In one embodiment, the digital image can be divided into a set of tiles. Tiling the image can include dividing the original image into smaller images that are easier to manage, called tiles. In one embodiment, the tiling operation is performed by applying a fixed grid to the whole-slide image, using a segmentation mask generated by a segmentation method, and selecting the tiles that contain tissue, or any other region of interest. In order to reduce the number of tiles to process even further, in one embodiment, additional or alternative selection methods can be used, such as random subsampling to keep only a given number of slides.

In one embodiment, augmentations may be applied to each of the sets of tiles. The process continues with generating a processed set of tiles by performing the following operations for each batch of tiles selected from the set of tiles. A first set of features is extracted from a first batch of augmented tiles, and a second set of features is extracted from a second batch of augmented tiles. In some embodiments, the augmented tiles include zoomed in or rotated views, or views with color augmentations. For example, since orientation is not important in histology slides, the slides can be rotated at various degrees. The slides can also be enlarged or zoomed in. The process then uses contrastive loss between pairs of the first and second set of extracted features in order to bring matching pairs of tiles closer and different pairs of tiles further apart. Contrastive loss is applied in order to pay attention to positive pairs taken from the first and second set of features, rather than negative pairs.

The process continues with training a feature extractor using the processed set of tiles generated via operations of the embodiments. In some embodiments, the classification of histology images can be improved using the trained feature extractor disclosed herein. The process continues with outputting a trained feature extractor that has been trained using a self-supervised ML algorithm. In some embodiments, a feature extractor can be trained for a particular number of epochs, so that each training image is seen a particular number of times.

With the trained machine learning model, the risk score can be computed. The process begins with receiving an input histology image. In some embodiments, the input histology image is a WSI, and it can be derived from a patient tissue sample. In some embodiments, the patient tissue sample is known or suspected to contain a tumor.

In some embodiments, the process includes removing background segments from the input image. In some embodiments, matter detection can be used to take only tiles from tissue regions of the input image. In some embodiments, the background can be removed using Otsu's method applied to the hue and saturation channels after transformation of the input image into hue, saturation, value (HSV) color space.

The process continues with tiling the histology image into a set of tiles. In one embodiment, the process uses the tiling to increase the ability of preprocessing the images. For example, and in one embodiment, using a tiling method is helpful in histopathology analysis, due to the large size of the whole-slide image. More broadly, when working with specialized images, such as histopathology slides, or satellite imagery, or other types of large images, the resolution of the image sensor used in these fields can grow as quickly as the capacity of random-access memory associated with the sensor. With this increased image size, it is difficult to store batches of images, or sometimes even a single image, inside the random-access memory of a computer. This difficulty is compounded if trying to store these large images in specialized memory of a Graphics Processing Unit (GPU). This situation makes it computationally intractable to process an image slide, or any other image of similar size, in its entirety.

In one embodiment, tiling the image (or the image minus the background) addresses this challenge by dividing the original image (or the image minus the background), into smaller images that are easier to manage, called tiles. In one embodiment, the tiling operation is performed by applying a fixed grid to the whole-slide image, using the segmentation mask generated by the segmentation method, and selecting the tiles that contain tissue, or any other kind of region of interest for the later classification process. As used herein, the “region of interest” of an image could be any region semantically relevant for the task to be performed, in particular regions corresponding to tissues, organs, bones, cells, body fluids, etc. when in the context of histopathology. In order to reduce the number of tiles to process even further, additional or alternative selection methods can be used, such as random subsampling to keep only a given number of slides.

For example, and in one embodiment, the process divides the image (or the image minus the background) into tiles of fixed size (e.g., each tile having a size of 774×774 pixels). Alternatively, the tile size can be smaller or larger. In this example, the number of tiles generated depends on the size of the matter detected and can vary from a few hundred tiles to 50,000 or more tiles. In one embodiment, the number of tiles is limited to a fixed number that can be set based on at least the computation time and memory requirements (e.g., 10,000 tiles).

For each tile, the process continues with extracting one or more features of that tile. In one embodiment, each of the features are extracted by applying a trained feature extractor that was trained with a contrastive loss ML algorithm using a training set of images. In one embodiment, the training set of images is a set of annotation-free images. In one embodiment, the input image and the training set of images are from the same domain, meaning that they are of the same category or type of image. For example, the input image and the training set of images can both be histology images. This is in contrast to an embodiment where the training set of images includes out-of-domain images, or images that are not histology images, or are not of the same category or type as the images being analyzed. In one embodiment, the contrastive loss ML algorithm is Momentum Contrast, or Momentum Contrast v7 (MoCo v7). In some embodiments, the trained feature extractor is an ImageNet type of feature extractor. In one embodiment, the trained machine learning model is the machine learning model as described herein.

In some embodiments, the machine learning model is a Deep Multiple Instance Learning model. In some embodiments, the machine learning model is a Weldon model. In some embodiments, the machine learning model is applied to the entire group of tiles. In some embodiments, the machine learning model is applied to a subset of tiles. The training images can include digital images of histologic sections of disease (e.g., cancer) samples derived from a number of control subjects. In some cases, the training images lack local annotations. The training images can include images associated with one or more global label(s) indicative of one or more disease feature(s) of the control patient from whom the sample is derived. The disease feature can include, in some embodiments, a duration of time of survival, a duration of time of disease-free survival, or a duration of time to disease (e.g., cancer) relapse. In some embodiments where the disease is cancer, the one or more disease feature(s) can include one or more of histological subtype, tumor stage, tumor size, number of positive lymph nodes, biomarker status, pathological staging, clinical staging, patient age, and/or treatment history, or a combination thereof. In some embodiments, one or more disease features of a patient can be obtained, and a machine learning model is applied to both the extracted features and the disease features of the patient. Example disease features of the patient can be the same as the disease features represented in the global labels associated with the training images.

The process then computes an AI risk score for the subject using the machine learning model. The AI risk score can represent the likelihood of a certain prognosis, e.g., overall survival, disease free survival, early relapse, or relapse following treatment, and may be expressed as a classification or a continuous range. In one embodiment, the AI risk score represents a predicted time from the diagnosis to death (overall survival) of the subject.

In some embodiments, a final risk score can be computed as a weighted average of the AI risk score and the clinical risk score, where the weights can be the same or different. With the trained machine learning and clinical models, the process can receive a WSI and clinical attributes to determine a risk score for a subject. In one embodiment, the WSI is a digital image associated with the subject and the one or more clinical attributes are attributes that are used as input for the clinical model. The process can then compute an AI risk score using the trained machine learning model, and compute a clinical risk score using the trained clinical model.

In one embodiment, the final risk score is an average of the machine learning and clinical risk scores. For example, and in one embodiment,

$${R}_{f} = \frac{{R}_{m} + {R}_{c}}{2}$$

where R_{f} is the final risk score, Rₘ is the AT risk score, and R_{c} is the machine clinical risk score. Alternatively, the final risk score can be a weighted average of the machine learning and clinical risk scores. For example, and in one embodiment,

$${R}_{f} = {a}_{m} {R}_{m} + {a}_{m} {R}_{m}$$

where aₘ and aₘ are the weights for Rₘ and R_{c}, respectively. In a further embodiment, the process can compute the final risk score differently (e.g., square root sum of the squares or another function of two inputs).

The final risk score can represent the likelihood of a certain prognosis, e.g., overall survival, disease free survival, early relapse, or relapse following treatment, and may be expressed as a classification or a continuous range. In one embodiment, the final risk score represents a predicted time from the diagnosis to death (overall survival) of the subject.

Deep learning model can surpass conventional biomarkers or histological classifications conventionally used by pathologists in prognostic prediction and can provide improvement in C-index when predicting clinical outcomes, such as overall survival (OS). For example, the deep learning model (HCCnet) was trained on 390 whole slide images (WSIs) obtained from 194 patients. HCCnet was then tested on 342 WSIs from 328 patients in the Cancer Genome Atlas (TCGA). In this validation set, the deep learning model outperformed a composite score. HCCnet obtained a C-index of 0.70 when the composite score only had a C-index of 0.63. This composite score was based on disease stage according to the American Joint Committee on Cancer (AJCC), age at diagnosis, sex, serum AFP, alcohol consumption, HBV (hepatitis B virus) or HCV infection, other etiologies, undetermined etiology, tumor differentiation according to the World Health Organization (WHO) criteria, macrovascular and microvascular invasion, positive surgical margins, and nontumoral liver fibrosis (cirrhosis).

### VI. Semi-Synthetic Simulation of Covariate Adjustment on Deep Learning Covariates Based on Clinical Trial Data

### A. Simulation on Hepatocellular Carcinoma (HCC) Data Using HCCnet

Patients with early stage hepatocellular carcinoma (HCC) are eligible for local treatment (resection or local ablation). No adjuvant treatment exists after resection or local ablation in HCC despite poor survival outcomes. In the STORM trial, sorafenib, the standard of care for advanced HCC, failed to show benefit over placebo in the adjuvant setting (Bruix et al. 2015 Lancet Oncol 2015; 16:1344-54). Large pharma-sponsored trials are currently investigating whether immunotherapy improves outcomes for patients with HCC after local treatment (clinicaltrials.gov NCT03383458, NCT04102098, NCT03867084, NCT03847428).

A deep-learning model on histological slides, HCCnet, captures an important prognostic signal on overall survival for HCC after curative treatment (Saillard et al. 2020 Hepatology 72:6; 2000-2013). In semi-synthetic simulations based on the original external validation set from TCGA-HCC (Cancer Genome Atlas Research Network 2017 Cell 169:1327-1341), reduction in sample size in adjuvant trials that could be obtained by adjustment on HCCnet was examined.

Eligibility criteria in clinical trials are too restrictive which leads to limited generalizability as well as difficulty in enrollment (Kim et al. 2017 J Clin Oncol Off J Am Soc Clin Oncol 35:3737-44; FDA 2020 https://www.fda.gov/regulatory-information/search-fda-guidance-documents/enhancing-diversity-clinical-trial-populations-eligibility-criteria-enrollment-practices-and-trial). Beyond ensuring patient safety, restrictive eligibility might be used to ensure homogeneity in the trial population (FDA 2018 Workshop Rep 12). In non-small cell lung cancer, it was shown using observational cohorts that many inclusion criteria are superfluous as they restrict the potential enrollment of trials even though the treatment is as efficacious for the excluded patients as for the included patients (Liu et al. 2021 Nature 592:629-633). As covariate adjustment allows to analytically compensate for the heterogeneity in the patient population, we investigated whether adequate covariate adjustment could allow to broaden eligibility criteria while maintaining statistical power. For this, we used both the parametric and the semi-synthetic (adjuvant HCC) simulation settings.

HCCnet was applied on 328 patients with early stage HCC from the TCGA HCC dataset (Saillard et al. 2020 Hepatology 72:6; 2000-2013; Cancer Genome Atlas Research Network 2017 Cell 169:1327-1341). Based on this dataset with the HCCnet predictions, we performed semi-synthetic simulations. In order to have a dataset with no missing data, we imputed all missing values among 73 clinical variables having less than 50% missing variables and more than one modality. We used a method relying on factorial analysis for mixed data (FAMD), a principal component method for data involving both continuous and categorical variables (Josse & Husson 2016 J Stat Softw 70). The imputed variables used as adjustment are tumor staging (1% missing values) and ECOG score which had 20% missing values. The imputed variables used as eligibility criteria are the ECOG score, the Child Pugh classification (33% missing), the macrovascular invasion (15% missing) and B or C hepatitis infection status (15% and 5% missing values respectively). Due to the large proportion of early censoring in the HCC-TCGA dataset, we assigned new event times while preserving the observed survival curve and dependence on covariates. To do so, a Cox model of overall survival is fitted on the available prognostic variables (tumor staging, ECOG score and the HCCnet variable). For each simulated patient, we sample her/his clinical covariates from TCGA. The hazard rate is defined as before except that 0 is replaced by d, the vector of coefficients from the Cox regression. The Weibull distribution is replaced by the empirical survival function that depends on the hazard rate and on the baseline survival function So from the Cox regression:

$$\{ \begin{matrix}{h}_{i} ( z ) = {hr}^{2} exp ( κ + {\hat{θ}}^{⊤} {X}_{i} ) , \\ \hat{S} ( t \vert z , {X}_{i} ) = {{\hat{S}}_{0} ( t )}^{{h}_{i} ( z )} , \\ {T}_{i} ( z ) \sim {\hat{S}}^{- 1} ( \cdot \vert z , {X}_{i} )\end{matrix} .$$

As before, κ is numerically optimized in order to set the incidence of outcome in the placebo arm to the observed incidence of outcome. Finally, all patients with events after 5 years are censored at that time. A sample size of 760 was selected for the simulated trial as it is the average sample size among 4 ongoing trials for adjuvant treatment in early stage HCC (clinicaltrials.gov NCT03383458, NCT04102098, NCT03867084, NCT03847428). The treatment effect size hr is set so that the estimated statistical power reached with adjustment on the clinical variables is 80% for this sample size. Randomization of the treatment assignment is stratified on tumor staging. The power curves of two choices of adjustment variables were compared: adjustment on clinical variables (tumor staging and ECOG score), and the adjustment on HCCnet as well as the clinical variables. The statistical power is estimated on 5e+3 resamples.

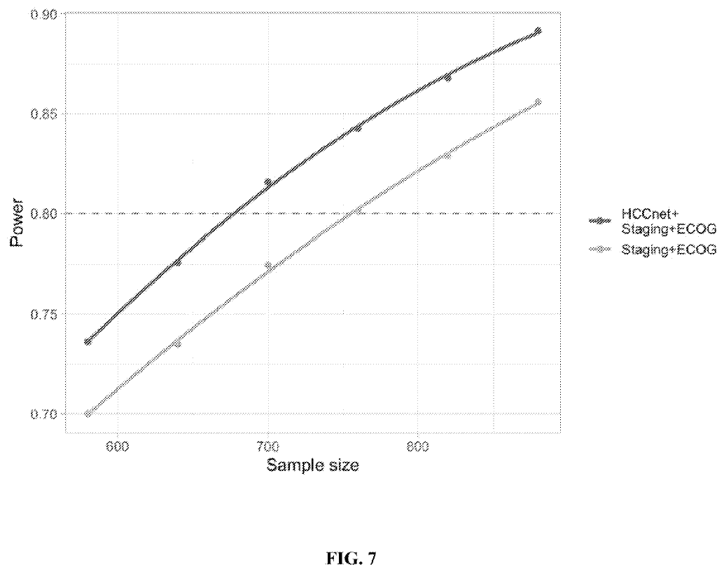

FIG. 7 shows the power curves when adding HCCnet to tumor staging and ECOG.

HCCnet allows a sample size reduction of R_{obs}²=10.3% and an increase of 4.4% in power, at power 80%.

The compatibility of this result with the results of the parametric simulations. The incidence of death in the HCC-TCGA population is 32.3% at 5 years. Tumor staging and ECOG score have a C-index of 0.65 in the source population, while adding HCCnet results in a C-index of 0.72.

As used herein, the quantities associated with adjustment on the clinical variables (tumor staging and ECOG) is labeled as 1, and the quantities associated with adjustment on the HCCnet model (tumor staging, ECOG, and HCCnet) is labeled as 2. Applying the Fleiss equation for the two adjustments using the R_{obs,i}² from FIG. 7A, the following result is obtained, demonstrating that adjustment on the HCCnet and clinical variables allows a sample size reduction of 10.9% as compared to adjustment on the clinical variables alone:

$${N}_{adj , 2} / {N}_{adj , 1} = ( 1 - {R}_{obs , 2}^{2} ) / ( 1 - {R}_{obs , 1}^{2} ) ≃ 0.82 / 0.92 = 0.891 = 100 \% - 10.9 \% .$$

Therefore, the semi-synthetic simulations are coherent with the findings of the parametric simulations. Moreover, computing the R_{CS}² measure on the TCGA population between the HCCnet and the clinical models gives a value of 9.0%.

### B. Simulation on Mesothelioma Data Using MesoNet

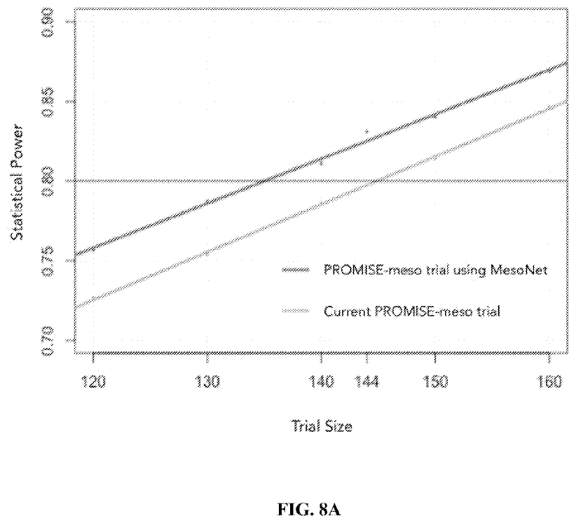

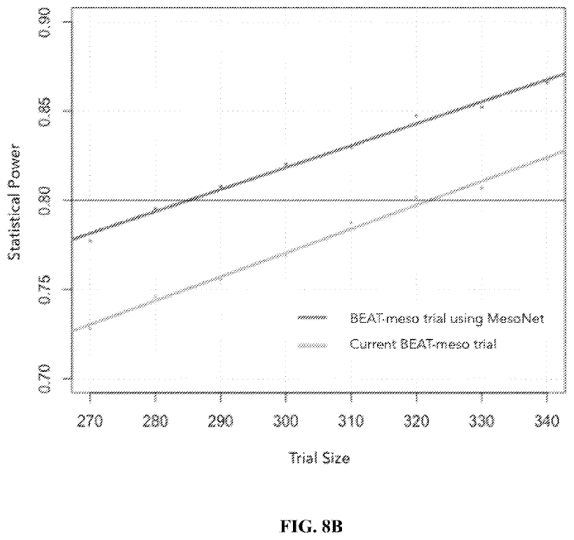

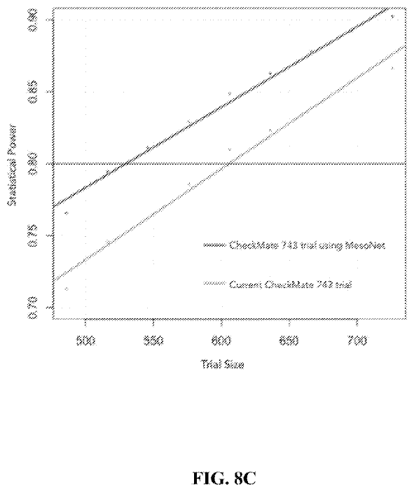

MesoNet, a deep learning model that predicts the overall survival of malignant mesothelioma patients using whole slide (histology) images of tumor tissue has been developed (Courtiol et al. 2019 Nat Medicine 25:10; 1519-1525). The deep learning survival predictions can outperform the existing subtype classification utilized by pathologists. As shown in FIGS. 8A-8C, the use of MesoNet risk scores as a covariate adjustment in clinical trial primary analysis in addition to histological subtype would allow researchers to reduce the sample size requirements of three, large phase 3 clinical trials in mesothelioma (PROMISE-meso trial, BEAT-meso trial, CheckMate 743 trial) by 6-13%. In these simulations, the current clinical trials are assumed to stratify patients and adjust the analysis on histological subtype (epithelioid vs. sarcomatoid vs. mixed) and to have a sample size corresponding to 80% statistical power. All of these simulations were run using the MesoNet training dataset from the Mesobank.

Furthermore, assuming that patient enrollment is conducted on a rolling basis, the decrease in sample size obtained using MesoNet would allow for a 2-8 month reduction in trial duration as compared to current practice. Therefore, leveraging MesoNet can reduce the sample size of mesothelioma trials, lowering costs, and reducing trial lengths.

### VII. Achieving Targeted Statistical Power Using Less Strict Eligibility Criteria

The present disclosure provides methods for achieving a targeted statistical power using less strict eligibility criteria in a trial by using covariate adjustment that can compensate for the increased heterogeneity that comes with less restrictive inclusion criteria. Using the parametric simulations and the semi-synthetic simulations, the interplay between more or less restrictive inclusion criteria and adjustment on covariates were considered. In the case of parametric simulations, the restricted inclusion criteria are based on the values of X, the covariate summarizing the prognostic information: only patients with X below 0, i.e. patients at lower risk (X is associated to a hazard ratio of 2 for this experiment) were included for the most restrictive eligibility criteria. There are then 4 scenarios when combining the two possible eligibility criteria (all patients or restricted inclusion) and the two choices of adjustments (no adjustment or adjustment on X). Other parameters are the Weibull shape and treatment hazard ratio which are set to 1, 0.7 respectively. The observed outcome incidence is 96.5% when all patients are included, and 93.5% for the restricted inclusion.

In the case of the HCC semi-synthetic simulations, we defined two additional levels of restricted eligibility criteria as shown in Table 2. The mildly restrictive eligibility level has two inclusion criteria present in all 4 ongoing large trials for adjuvant treatment in early stage HCC (clinicaltrials.gov NCT03383458, NCT04102098, NCT03867084, NCT03847428): only patients with a Child-Pugh score of A and with an ECOG status of 0 or 1 are included. The most restrictive eligibility criteria further restrict the ECOG status to 0 as in the STORM trial (Bruix et al. 2015 Lancet Oncol 16:1344-54), excludes patients with a dual infection of hepatitis B and hepatitis C as in the KEYNOTE-937 trial (clinicaltrials.gov NCT03867084) and excludes patients with macrovascular invasion as in the IMBRAVE050 trial (clinicaltrials.gov NCT04102098). We considered only the eligibility criteria that were available in the TCGA HCC dataset. There are therefore 6 different scenarios when combining the two choices of adjustment considered (clinical adjustment: tumor staging and ECOG or HCCnet adjustment) and the three levels of eligibility. In the scenario with the most restrictive eligibility levels, every patient has an ECOG of 0 and therefore the analyses were not adjusted on ECOG.

In both cases, changing the inclusion criteria changes the number of events which affects statistical power directly. Consequently, the statistical power of the different scenarios is presented as a function of the number of events. In both cases, no drop-out was added and 5000 repetitions were performed.

TABLE 2
Definition of eligibility levels from the HCC-TCGA dataset

| Eligibility level | Nested inclusion criteria | N (%) | |
|---|---|---|---|
| Less restrictive | All patients included in the validation of | 328 | (100%) |
| | HCCnet on TCGA | | |
| Mildly | Child Pugh classification is A | 275 | (84%) |
| restrictive | ECOG ≤ 1 | | |
| Most restrictive | ECOG score of 0 | 180 | (55%) |
| | No macrovascular invasion | | |
| | No cumulated hepatitis B and C | | |
| | infection | | |

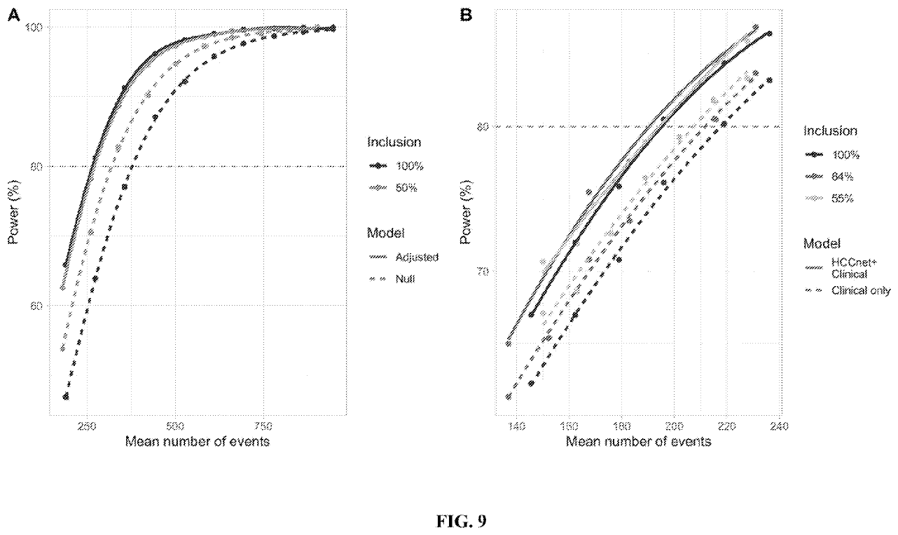

FIG. 9A depicts interplay between eligibility criteria and choice of adjustment in parametric simulations where the least at risk patients are selected in the 50% inclusion. FIG. 9B depicts interplay between eligibility criteria and choice of adjustment in the semi-synthetic simulation based on the HCC-TCGA dataset. FIGS. 9A and 9B show that, both in parametric simulations and semi-synthetic simulations, restricting the population allows one to have more statistical power for the same number of events with an unadjusted analysis. This is due to the increased homogeneity of the population. However, when the analysis is adjusted using all prognostic information, the same power curve can be obtained regardless of the inclusion (eligibility) criteria. Patients at lower risk of the event (e.g., death) are selected for enrollment when more restrictive criteria are considered. Accordingly, considering sample size (i.e., inclusive of subjects who have events and subjects who do not have events) instead of the number of events would favor the more inclusive criteria.

While the adjusted analyses with different eligibility criteria have the same statistical power, they imply a very different size of screened population. For instance, in the HCC example, the required size of the screened population is 604 for the less restrictive inclusion while it is 1729 for the most restrictive population. Therefore, the screened population is reduced by 65% while attaining the same statistical power. This difference in screened population is explained by the smaller proportion of patients included as well as the smaller proportion of events with the restrictive eligibility criteria (20.5% at 5 years versus 32.3% in the entire population).

Restrictive eligibility criteria ensure homogeneous populations but lead to difficulty in enrollment as well as questionable generalizability of trial results. This has led to calls for less restrictive eligibility criteria (FDA 2020 https://www.fda.gov/regulatory-information/search-fda-guidance-documents/enhancing-diversity-clinical-trial-populations-eligibility-criteria-enrollment-practices-and-trial; FDA 2018 Workshop Rep 12). The present disclosure provides that adequate covariate adjustment removes any incentive to homogenize the population using restrictive eligibility criteria. Indeed, in both parametric simulations and semi-synthetic simulations based on the actual clinical trial dataset, the adjusted analysis is just as powerful regardless of strictness of eligibility criteria. Accordingly, the size of the population that needs to be screened for inclusion can be reduced substantially by using the less restrictive eligibility criteria while maintaining the same statistical power by performing covariate adjustment.

As noted in the draft FDA guidance, covariate adjustment changes the target of estimation, a phenomenon called non-collapsibility (FDA 2021 https://www.fda.gov/regulatory-information/search-fda-guidance-documents/adjusting-covariates-randomized-clinical-trials-drugs-and-biological-products). If a marginal estimand is preferred, one can consider adjusted marginal estimators that target the estimand of the unadjusted analysis while leveraging the gain in precision offered by covariates (Daniel et al. 2020 Biom J 63:528-557; Permutt 2020 Stat Biopharm Res 12:45-53).

The methods described herein encompass adjustment covariates associated with relative measures of treatment effect (e.g., the hazard ratio) as well as absolute measures of efficacy, such as restricted mean survival time or absolute risk reduction. Both relative and absolute measures of treatment efficacy can be made more precise with the prognostic signal of covariates according to the present disclosure.

### VIII. Computer System and Machine Readable Medium

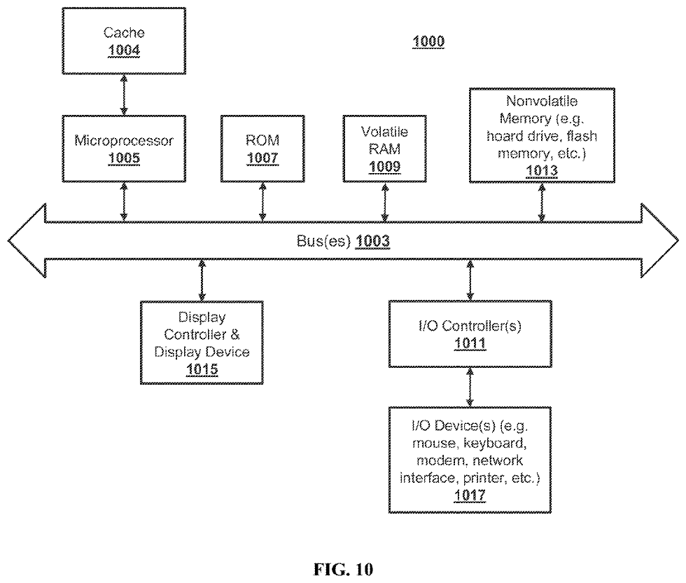

As shown in FIG. 10, the computer system 1000, which is a form of a data processing system, includes a bus 1003 which is coupled to a microprocessor(s) 1005 and a ROM (Read Only Memory) 1007 and volatile RAM 1009 and a non-volatile memory 1013. The microprocessor 1005 may include one or more CPU(s), GPU(s), a specialized processor, and/or a combination thereof. The microprocessor 1005 may be in communication with a cache 1004, and may retrieve the instructions from the memories 1007, 1009, 1013 and execute the instructions to perform operations described above. The bus 1003 interconnects these various components together and also interconnects these components 1005, 1007, 1009, and 1013 to a display controller and display device 1015 and to peripheral devices such as input/output (I/O) devices 1011 which may be mice, keyboards, modems, network interfaces, printers and other devices which are well known in the art. Typically, the input/output devices 1011 are coupled to the system through input/output controllers 1017. The volatile RAM (Random Access Memory) 1009 is typically implemented as dynamic RAM (DRAM), which requires power continually in order to refresh or maintain the data in the memory.

The nonvolatile memory 1013 can be, for example, a magnetic hard drive or a magnetic optical drive or an optical drive or a DVD RAM or a flash memory or other types of memory systems, which maintain data (e.g. large amounts of data) even after power is removed from the system. Typically, the nonvolatile memory 1013 will also be a random access memory although this is not required. While FIG. 10 shows that the nonvolatile memory 1013 is a local device coupled directly to the rest of the components in the data processing system, it will be appreciated that the present invention may utilize a nonvolatile memory which is remote from the system, such as a network storage device which is coupled to the data processing system through a network interface such as a modem, an Ethernet interface or a wireless network. The bus 1003 may include one or more buses connected to each other through various bridges, controllers and/or adapters as is well known in the art.

Portions of what was described above may be implemented with logic circuitry such as a dedicated logic circuit or with a microcontroller or other form of processing core that executes program code instructions. Thus processes taught by the discussion above may be performed with program code such as machine-executable instructions that cause a machine that executes these instructions to perform certain functions. In this context, a “machine” may be a machine that converts intermediate form (or “abstract”) instructions into processor specific instructions (e.g., an abstract execution environment such as a “virtual machine” (e.g., a Java Virtual Machine), an interpreter, a Common Language Runtime, a high-level language virtual machine, etc.), and/or, electronic circuitry disposed on a semiconductor chip (e.g., “logic circuitry” implemented with transistors) designed to execute instructions such as a general-purpose processor and/or a special-purpose processor. Processes taught by the discussion above may also be performed by (in the alternative to a machine or in combination with a machine) electronic circuitry designed to perform the processes (or a portion thereof) without the execution of program code.

The present invention also relates to an apparatus for performing the operations described herein. This apparatus may be specially constructed for the required purpose, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, and magnetic-optical disks, read-only memories (ROMs), RAMs, EPROMs, EEPROMs, magnetic or optical cards, or any type of media suitable for storing electronic instructions, and each coupled to a computer system bus.

A machine readable medium includes any mechanism for storing or transmitting information in a form readable by a machine (e.g., a computer). For example, a machine readable medium includes read only memory (“ROM”); random access memory (“RAM”); magnetic disk storage media; optical storage media; flash memory devices; etc.

An article of manufacture may be used to store program code. An article of manufacture that stores program code may be embodied as, but is not limited to, one or more memories (e.g., one or more flash memories, random access memories (static, dynamic or other)), optical disks, CD-ROMs, DVD ROMs, EPROMs, EEPROMs, magnetic or optical cards or other type of machine-readable media suitable for storing electronic instructions. Program code may also be downloaded from a remote computer (e.g., a server) to a requesting computer (e.g., a client) by way of data signals embodied in a propagation medium (e.g., via a communication link (e.g., a network connection)).

The preceding detailed descriptions are presented in terms of algorithms and symbolic representations of operations on data bits within a computer memory. These algorithmic descriptions and representations are the tools used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be kept in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “segmenting,” “tiling,” “receiving,” “computing,” “extracting,” “processing,” “applying,” “augmenting,” “normalizing,” “pre-training,” “sorting,” “selecting,” “aggregating,” “sorting,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The processes and displays presented herein are not inherently related to any particular computer or other apparatus. Various general-purpose systems may be used with programs in accordance with the teachings herein, or it may prove convenient to construct a more specialized apparatus to perform the operations described. The required structure for a variety of these systems will be evident from the description herein. In addition, the present invention is not described with reference to any particular programming language. It will be appreciated that a variety of programming languages may be used to implement the teachings of the invention as described herein.

The foregoing discussion merely describes some exemplary embodiments of the present invention. One skilled in the art will readily recognize from such discussion, the accompanying drawings and the claims that various modifications can be made without departing from the spirit and scope of the invention. Furthermore, where feasible, any of the aspects disclosed herein may be combined with each other (e.g., the feature according to one aspect may be added to the features of another aspect or replace an equivalent feature of another aspect) or with features that are well known in the art, unless indicated otherwise by context.

All citations to references, including, for example, citations to patents, published patent applications, and articles, are herein incorporated by reference in their entirety.

The section headings used herein are for organizational purposes only and are not to be construed as limiting the subject matter described in any way.

What is claimed is:

1. A method for designing a randomized controlled trial (RCT) with a time-to-event outcome, said method comprising:
selecting a covariate for adjustment; and
calculating a number of events required to obtain a statistical power based on a formula:
N_{adjusted}=N_{original}(1−R_{CS}²); wherein
the RCT is conducted using the calculated number of events,
wherein the N_{original} is an original number of events required to obtain the statistical power without covariate adjustment,
wherein the N_{adjusted} is an adjusted number of events required to obtain the statistical power with covariate adjustment, and
wherein the R_{CS}² is computed on data external to the RCT based on a formula:
$${R}_{CS}^{2} = 1 - exp [ - \frac{2}{n} ( {l}_{1} - {l}_{0} ) ] ,$$
wherein
the R_{CS}² is a Cox-Snell R²,
the n is a number of participants,
the l₀ is a log-likelihood of a Cox model to explain the time-to-event outcome with an intercept only, and
the l₁ is a log-likelihood of a Cox model to explain the time-to-event outcome with an intercept and with covariate adjustment.

2. A method for evaluating sample size at an interim stage of an ongoing randomized controlled trial (RCT), said method comprising:
selecting a covariate for adjustment;
obtaining blinded RCT data; and
performing a blinded sample size reestimation, at the interim stage, using R_{CS}² and a formula:
N_{adjusted}=N_{original}(1−R_{CS}²); wherein
the RCT is further conducted using the blinded sample size reestimation,
wherein the N_{original} is an original number of events required to obtain the statistical power without covariate adjustment,
wherein the N_{adjusted} is a reestimated number of events required to obtain the statistical power;
wherein the R_{CS}² is computed, at the interim stage, on the blinded RCT data based on a formula:
$${R}_{CS}^{2} = 1 - exp [ - \frac{2}{n} ( {l}_{1} - {l}_{0} ) ] ,$$
wherein
the R_{CS}² is a Cox-Snell R²,
the n is a number of participants,
the l₀ is a log-likelihood of a Cox model to explain the time-to-event outcome with an intercept only, and
the l₁ is a log-likelihood of a Cox model to explain the time-to-event outcome with an intercept and with covariate adjustment.

3. The method of claim 1 or 2, further comprising evaluating the original number of events required to obtain the statistical power without covariate adjustment (N_{original}) based on a formula:
N_{original}=(z_{β}+z_{1-α})²/(P₁P₂ log² (hr)), wherein
the N_{original} is an estimated number of events required to obtain the statistical power based on the Schoenfeld formula,
the α is a type I error level,
the β is a type II error level,
the P₁ and the P₂ are the proportion of the trial sample included in the treatment and control arm respectively (e.g. both are equal to ½ if the treatment allocation is balanced),
the hr is a stipulated hazard ratio, and
the zₚ is a p-quantile of the standard normal distribution.

4. The method of any one of claims 1-3, wherein the time-to-event outcome is overall survival, disease free survival, or time to disease relapse.

5. The method of any one of claims 1-4, wherein the RCT is conducted to evaluate a treatment effect in cancer patients.

6. The method of claim 5, wherein the cancer is hepatocellular carcinoma, mesothelioma, pancreatic cancer, lung cancer, or breast cancer.

7. The method of any one of the claims 1-6, wherein covariate adjustment is conducted on a clinical risk score, said method comprising:
obtaining clinical attributes derived from the subject;
computing a clinical risk score using a clinical model, the clinical model trained using one or more subject attribute,
wherein the clinical risk score quantifies the prognosis of the subject.

8. The method of any one of claims 1-6, wherein covariate adjustment is conducted on a covariate obtained by a deep learning model.

9. The method of claim 8, wherein the deep learning model is based on histopathological slides obtained from cancer subjects, and the covariate is a prognostic covariate.

10. The method of claim 8 or 9, wherein the covariate is obtained by a computer-implemented method for determining a likelihood of prognosis of a subject having a disease, comprising:
accessing a digital histology image of a histology section obtained from the subject;
extracting a plurality of feature vectors of the histology image by applying a first convolutional neural network, wherein each of the features of the plurality of feature vectors represents local descriptors of the histology image;
classifying the histology image using at least the plurality of feature vectors and a classification model, wherein the classification model is trained using a training set of known histology images and known prognosis information; and
determining the likelihood of prognosis of the subject based on at least the classification of the histology image.

11. The method of claim 8 or 9, wherein the covariate is obtained by a computer-implemented method for determining the prognosis of a subject having a disease, said method comprising:
obtaining a digital histology image of a histology section from the subject;
dividing the digital image into a set of tiles;
extracting a plurality of feature vectors from the set of tiles, or a subset thereof; and
computing an artificial intelligence (AI) risk score based on the histology image using a machine learning model, the machine learning model having been trained by processing a plurality of training images to predict the prognosis,
wherein the Al risk score quantifies the prognosis of the subject.

12. The method of claim 11, further comprising:
obtaining clinical attributes derived from the subject;
computing a clinical risk score using a clinical model, the clinical model trained using one or more subject attributes; and
computing a final risk score for the subject from the Al risk score and the clinical risk score,
wherein the final risk score quantifies the prognosis of the subject.

13. The method of any one of claims 10-12, wherein the digital histology image is a whole slide image (WSI).

14. The method of any one of claims 10-13, wherein the histology section has been stained with a dye.

15. The method of claim 14, wherein the dye is hematoxylin and eosin (H&E).

16. The method of any one of 10-15, wherein the disease is cancer.

17. The method of claim 16, wherein the cancer is hepatocellular carcinoma, mesothelioma, pancreatic cancer, lung cancer, or breast cancer.

18. The method of any one of claims 1-17, wherein subject enrollment based on restrictive eligibility criteria do not improve statistical power relative to subject enrollment based on less restrictive eligibility criteria.

19. The method of any one of claims 1-18, wherein a targeted statistical power is achieved using less strict eligibility criteria in a trial.

20. The method of any one of claims 1-19, wherein the method is implemented by a computer.

21. A machine readable medium having executable instructions to cause one or more processing units to perform a method of designing a randomized controlled trial (RCT), the method comprising:
selecting a covariate for adjustment; and
calculating a sample size required to obtain a statistical power based on a formula:
N_{adjusted}=N_{original} (1−R_{CS}²); wherein
the RCT is conducted using the calculated sample size,
wherein the N_{original} is an original number of events required to obtain the statistical power without covariate adjustment,
wherein the N_{adjusted} is an adjusted number of events required to obtain the statistical power with covariate adjustment, and
wherein the R_{CS}² is computed on data external to the RCT based on a formula:
$${R}_{CS}^{2} = 1 - exp [ - \frac{2}{n} ( {l}_{1} - {l}_{0} ) ] ,$$
wherein
the R_{CS}² is a Cox-Snell R²,
the n is a number of participants,
the l₀ is a log-likelihood of a Cox model to explain the time-to-event outcome with an intercept only, and
the l₁ is a log-likelihood of a Cox model to explain the time-to-event outcome with an intercept and with covariate adjustment.

22. A machine readable medium having executable instructions to cause one or more processing units to perform a method of evaluating sample size required to obtain a statistical power at an interim stage of an ongoing randomized controlled trial (RCT), the method comprising:
selecting a covariate for adjustment;
obtaining blinded RCT data; and
performing a blinded sample size reestimation, at the interim stage, using R_{CS}² and a formula:
N_{adjusted}=N_{original}(1−R_{CS}²); wherein
the RCT is further conducted using the blinded sample size reestimation,
wherein the N_{original} is an original number of events required to obtain the statistical power without covariate adjustment,
wherein the N_{adjusted} is a reestimated number of events required to obtain the statistical power;
wherein the R_{CS}² is computed, at the interim stage, on the blinded RCT data based on a formula:
$${R}_{CS}^{2} = 1 - exp [ - \frac{2}{n} ( {l}_{1} - {l}_{0} ) ] ,$$
wherein
the R_{CS}² is a Cox-Snell R²,
the n is a number of participants,
the l₀ is a log-likelihood of a Cox model to explain the time-to-event outcome with an intercept only, and
the l₁ is a log-likelihood of a Cox model to explain the time-to-event outcome with an intercept and with covariate adjustment.

23. The machine readable medium of claim 21 or 22, wherein the method further comprises evaluating the original number of events required to obtain the statistical power without covariate adjustment (N_{original}) based on a formula:
N_{original}=(z_{β}+z_{1-α)}²/(P₁P₂ log² (hr)), wherein
the N_{original} is an estimated number of events required to obtain the statistical power based on the Schoenfeld formula,
the α is a type I error level,
the β is a type II error level,
the P₁ and the P₂ are the proportion of the trial sample included in the treatment and control arm respectively (e.g. both are equal to 12 if the treatment allocation is balanced), and
the hr is a stipulated hazard ratio, and
the zₚ is a p-quantile of the standard normal distribution.

24. The machine readable medium of any one of claims 21-23, wherein the time-to-event outcome is overall survival, disease free survival, or time to disease relapse.

25. The machine readable medium of any one of claims 21-24, wherein the RCT is conducted to evaluate a treatment effect in cancer patients.

26. The machine readable medium of claim 25, wherein the cancer is hepatocellular carcinoma, mesothelioma, pancreatic cancer, lung cancer, or breast cancer.

27. The machine readable medium of any one of the claims 21-26, wherein covariate adjustment is conducted on a clinical risk score, said machine readable medium comprising:
obtaining clinical attributes derived from the subject;
computing a clinical risk score using a clinical model, the clinical model trained using one or more subject attribute,
wherein the clinical risk score quantifies the prognosis of the subject.

28. The machine readable medium of any one of claims 21-26, wherein covariate adjustment is conducted on a covariate obtained by a deep learning model.

29. The machine readable medium of claim 28, wherein the deep learning model is based on histopathological slides obtained from cancer subjects, and the covariate is a prognostic covariate.

30. The machine readable medium of claim 28 or 29, wherein the covariate is obtained by a computer-implemented machine readable medium for determining a likelihood of prognosis of a subject having a disease, comprising:
accessing a digital histology image of a histology section obtained from the subject;
extracting a plurality of feature vectors of the histology image by applying a first convolutional neural network, wherein each of the features of the plurality of feature vectors represents local descriptors of the histology image;
classifying the histology image using at least the plurality of feature vectors and a classification model, wherein the classification model is trained using a training set of known histology images and known prognosis information; and
determining the likelihood of prognosis of the subject based on at least the classification of the histology image.

31. The machine readable medium of claim 28 or 29, wherein the covariate is obtained by a computer-implemented machine readable medium for determining the prognosis of a subject having a disease, said machine readable medium comprising:
obtaining a digital histology image of a histology section from the subject;
dividing the digital image into a set of tiles;
extracting a plurality of feature vectors from the set of tiles, or a subset thereof; and
computing an artificial intelligence (AI) risk score based on the histology image using a machine learning model, the machine learning model having been trained by processing a plurality of training images to predict the prognosis,
wherein the Al risk score quantifies the prognosis of the subject.

32. The machine readable medium of claim 31, further comprising:
obtaining clinical attributes derived from the subject;
computing a clinical risk score using a clinical model, the clinical model trained using one or more subject attributes; and
computing a final risk score for the subject from the Al risk score and the clinical risk score,
wherein the final risk score quantifies the prognosis of the subject.

33. The machine readable medium of any one of claims 30-32, wherein the digital histology image is a whole slide image (WSI).

34. The machine readable medium of any one of claims 30-33, wherein the histology section has been stained with a dye.

35. The machine readable medium of claim 34, wherein the dye is hematoxylin and eosin (H&E).

36. The machine readable medium of any one of claims 30-35, wherein the disease is cancer.

37. The machine readable medium of claim 36, wherein the cancer is hepatocellular carcinoma, mesothelioma, pancreatic cancer, lung cancer, or breast cancer.

38. The machine readable medium of any one of claims 21-37, wherein subject enrollment based on restrictive eligibility criteria do not improve statistical power relative to subject enrollment based on less restrictive eligibility criteria.

39. The machine readable medium of any one of claims 21-38, wherein a targeted statistical power is achieved using less strict eligibility criteria in a trial.
